(19) United States
(12) Patent Application Publication
(10) Pub. No.: US 2015/0373999 A1
(43) Pub. Date: Dec. 31, 2015

(54) COMBINATION OF AN ALPHA-AMYLASE AND A G4-FORMING AMYLASE

(71) Applicant: DSM IP ASSETS B.V., Heerlen (NL)

(72) Inventors: Caroline Hendrine Maria VAN BENSCHOP, Echt (NL); Arie Gerrit TERDU, Echt (NL)

(21) Appl. No.: 14/767,410

(22) PCT Filed: Feb. 27, 2014
(86) PCT No.: PCT/EP2014/053886

§ 371 Date: Aug. 12, 2015

Related U.S. Application Data
(60) Provisional application No. 61/771,668, filed on Mar. 1, 2013.

(30) Foreign Application Priority Data
Mar. 1, 2013 (EP) 13157397.4

(51) Int. Cl. A21D 8/04; C12N 9/26; A21D 10/00; C12N 9/28

(52) U.S. Cl. CPC A21D 8/042; C12N 9/2417; C12N 9/2411; A21D 10/005; C12Y 302/01001; C12Y 302/0106

(57) ABSTRACT

This invention relates to an enzyme composition comprising an alpha-amylase polypeptide and a G4-forming amylase, a pre-mix comprising these enzymes, a method to prepare a dough and a method to prepare a baked product.

The invention also relates to methods of using the enzyme composition and the pre-mix in industrial processes, for example in food industry, such as the baking industry. The invention further relates to use of the enzyme composition or the pre-mix to reduce hardness after storage of a baked and/or to reduce loss of resilience over storage of a baked product.

### FIELD OF THE INVENTION

The present invention relates to an enzyme composition comprising an alpha-amylase polypeptide and a G4-forming amylase, a pre-mix comprising these enzymes, a method to prepare a dough and a method to prepare a baked product.

The invention also relates to methods of using the enzyme composition and the pre-mix in industrial processes, for example in food industry, such as the baking industry. The invention further relates to use of the enzyme composition or the pre-mix to reduce hardness after storage of a baked product and/or to reduce loss of resilience over storage of a baked product.

### BACKGROUND OF THE INVENTION

Studies on bread staling have indicated that the starch fraction in bread recrystallizes during storage, thus causing an increase in crumb firmness, which may be measured as an increase in hardness of bread slices. Reduction of staling of baked products, such as bread and cake, has been a point of attention in the food-industry.

U.S. Pat. No. 4,598,048 describes the preparation of a maltogenic amylase enzyme. U.S. Pat. No. 4,604,355 describes a maltogenic amylase enzyme, preparation and use thereof. U.S. RE38,507 describes an antistaling process and agent.

WO2008/148845 describes a METHOD OF PREPARING A DOUGH-BASED PRODUCT

WO2006/032281 describes a METHOD OF PREPARING A DOUGH-BASED PRODUCT

WO9950399 describes NON-MALTOGENIC EXOAMYLASES AND THEIR USE IN RETARDING RETROGRADATION OF STARCH.

WO2005007818 describes EXO-SPECIFIC AMYLASE POLYPEPTIDES, NUCLEIC ACIDS ENCODING THOSE POLYPEPTIDES AND USES THEREOF.

WO2004111217 describes a VARIANT PSEUDOMONAS POLYPEPTIDES HAVING A NON-MALTOGENIC EXOAMYLASE ACTIVITY AND THEIR USE IN PREPARING FOOD PRODUCTS

WO2005003339 describes FOOD ADDITIVE COMPRISING PSEUDOMONAS NON-MALTOGENIC EXOAMYLASE VARIANTS

WO2005007818 describes EXO-SPECIFIC AMYLASE POLYPEPTIDES, NUCLEIC ACIDS ENCODING THOSE POLYPEPTIDES AND USES THEREOF

WO2005007867 describes THERMOSTABLE AMYLASE POLYPEPTIDES, NUCLEIC ACIDS ENCODING THOSE POLYPEPTIDES AND USES THEREOF WO2006003461 describes a POLYPEPTIDE

WO2007007053 describes MODIFIED AMYLASE FROM PSEUDOMONAS SACCHAROPHILIA

WO2007148224 describes a polypeptide WO2009083592 describes PSEUDOMONAS SACCHAROPHILA G4-AMYLASE VARIANTS AND USES THEREOF

WO2009088465 describes A PROCESS OF OBTAINING ETHANOL WITHOUT GLUCOAMYLASE USING PSEUDOMONAS SACCHAROPHILA G4-AMYLASE AND VARIANTS THEREOF

WO2010133644 describes AMYLASE POLYPEPTIDES

WO2010118269 describes the production of maltotetraosesyrup using a PSEUDOMONAS SACCHAROPHILIA maltotetraohydrolase variant.

WO2010132157 describes the production of maltotetraosesyrup using a PSEUDOMONAS SACCHAROPHILIA maltotetraohydrolase variant and a debranching enzyme.

There is a need in the industry to further improve properties of dough and or baked products made from such dough.

### SUMMARY OF THE INVENTION

The present invention relates to a process to prepare a dough comprising adding an alpha-amylase polypeptide and a G-4 forming amylase.

The method to prepare a dough according to the invention comprises combining

i) an alpha-amylase polypeptide comprising

(a) an amino acid sequence as set out in amino acids 34 to 719 of SEQ ID NO: 2; or

(b) an amino acid sequence having at least 99.5% identity to an amino acid sequence as set out in amino acids 34 to 719 of SEQ ID NO: 2; or

(c) an amino acid sequence encoded by a polynucleotide as set out in nucleotides 100 to 2157 of SEQ ID NO: 1 or SEQ ID NO: 3; or

(d) an amino acid sequence having at least 70% identity to an amino acid sequence as set out in amino acids 34 to 719 of SEQ ID NO: 2 and having at least one of Asp at position 184, Ala at position 297, Thr at position 368 and Asn at position 489, said positions being defined with reference to SEQ ID NO: 2; or

(e) an amino acid sequence having at least 70% identity to an amino acid sequence as set out in amino acids 34 to 719 of SEQ ID NO: 2 and having at least one of Asp at position 184, Ala at position 297, Thr at position 368 and Asn at position 489, said positions being defined with reference to SEQ ID NO: 2 and said amino acid sequence characterized in that when used to prepare a baked product having at least 5 wt % sugar based on flour, said baked product has reduced hardness after storage in comparison with a baked product prepared without use of said amino acid sequence; or

(f) an amino acid sequence having alpha-amylase activity and having at least 70% identity to an amino acid sequence as set out in amino acids 34 to 719 of SEQ ID NO: 2 and having a substitution, at any one or more positions corresponding to 37, 39, 46, 47, 48, 49, 53, 78, 80, 84, 87, 94, 101, 102, 103, 104, 105, 106, 107, 108, 110, 111, 113, 115, 120, 121, 127, 128, 130, 133, 136, 137, 150, 157, 158, 159, 161, 162, 163, 166, 167, 169, 176, 177, 179, 201, 207, 210, 211, 216, 219, 221, 222, 223, 227, 228, 232, 233, 234, 237, 240, 243, 247, 250, 252, 255, 258, 260, 266, 267, 268, 269, 273, 284, 285, 287, 291, 292, 293, 295, 296, 297, 299, 300, 302, 304, 306, 312, 314, 315, 316, 317, 319, 321, 332, 355, 356, 358, 360, 361, 364, 367, 383, 389, 391, 400, 403, 404, 407, 410, 411, 421, 424, 447, 454, 455, 478, 483, 500, 521, 538, 569, 581, 616, 621, 636, 670, 681, 684, 685, 693, 709, 710,

said positions being defined with reference to an amino acid sequence as set out in amino acids 34 to 719 of SEQ ID NO: 2;

and

ii) a G4-forming amylase having an amino acid sequence at least 70% identical to the amino acid sequence as set out in SEQ ID NO: 4; and
iii) at least one dough ingredient.

The invention further relates to an enzyme composition that may be used for retarding staling of baked products such as bread and cake. Accordingly, the invention relates to an enzyme composition comprising the alpha-amylase polypeptide and the G4-forming amylase. Accordingly the invention provides:

An enzyme composition comprising an alpha-amylase polypeptide comprising

(a) an amino acid sequence as set out in amino acids 34 to 719 of SEQ ID NO: 2; or

(b) an amino acid sequence having at least 99.5% identity to an amino acid sequence as set out in amino acids 34 to 719 of SEQ ID NO: 2; or

(c) an amino acid sequence encoded by a polynucleotide as set out in nucleotides 100 to 2157 of SEQ ID NO: 1 or SEQ ID NO: 3; or

(d) an amino acid sequence having at least 70% identity to an amino acid sequence as set out in amino acids 34 to 719 of SEQ ID NO: 2 and having at least one of Asp at position 184, Ala at position 297, Thr at position 368 and Asn at position 489, said positions being defined with reference to SEQ ID NO: 2; or

(e) an amino acid sequence having at least 70% identity to an amino acid sequence as set out in amino acids 34 to 719 of SEQ ID NO: 2 and having at least one of Asp at position 184, Ala at position 297, Thr at position 368 and Asn at position 489, said positions being defined with reference to SEQ ID NO: 2 and said amino acid sequence characterized in that when used to prepare a baked product having a least 5 wt % sugar based on flour, said baked product has reduced hardness after storage in comparison with a baked product prepared without use of said amino acid sequence; or

(f) an amino acid sequence having alpha-amylase activity and having at least 70% identity to an amino acid sequence as set out in amino acids 34 to 719 of SEQ ID NO: 2 and having a substitution, at any one or more positions corresponding to 37, 39, 46, 47, 48, 49, 53, 78, 80, 84, 87, 94, 101, 102, 103, 104, 105, 106, 107, 108, 110, 111, 113, 115, 120, 121, 127, 128, 130, 133, 136, 137, 150, 157, 158, 159, 161, 162, 163, 166, 167, 169, 176, 177, 179, 201, 207, 210, 211, 216, 219, 221, 222, 223, 227, 228, 232, 233, 234, 237, 240, 243, 247, 250, 252, 255, 258, 260, 266, 267, 268, 269, 273, 284, 285, 287, 291, 292, 293, 295, 296, 297, 299, 300, 302, 304, 306, 312, 314, 315, 316, 317, 319, 321, 332, 355, 356, 358, 360, 361, 364, 367, 383, 389, 391, 400, 403, 404, 407, 410, 411, 421, 424, 447, 454, 455, 478, 483, 500, 521, 538, 569, 581, 616, 621, 636, 670, 681, 684, 685, 693, 709, 710,

said positions being defined with reference to an amino acid sequence as set out in amino acids 34 to 719 of SEQ ID NO: 2;
and wherein the composition further comprises a G4-forming amylase having an amino acid sequence at least 70% identical to the amino acid sequence as set out in SEQ ID NO: 4.

Further described are novel alpha-amylase polypeptides.

Further the invention concerns a pre-mix comprising said alpha-amylase polypeptide and said G-4 forming amylase. The invention also relates to a dough comprising said alpha-amylase polypeptide and said G-4 forming amylase.

The invention also relates to a method to prepare a baked product comprising the step of baking the dough according to the invention.

The invention further relates to a baked product.

### DESCRIPTION OF FIGURES

FIG. 1. Foto illustrating foldability of a slice of bread manufactured without Mature DSM-AM (alpha-amylase polypeptide) and without PowerFRESH Special (G4-forming amylase product by DuPont Industrial Biosciences, Denmark).

FIG. 2. Foto illustrating of foldability of a slice of bread manufactured with 50 ppm Mature DSM-AM.

FIG. 3. Foto illustrating of foldability of a slice of bread manufactured with 75 ppm PowerFRESH Special.

FIG. 4. Foto illustrating of foldability of a slice of bread manufactured with 50 ppm Mature DSM-AM and 75 ppm PowerFRESH Special.

### BRIEF DESCRIPTION OF THE SEQUENCE LISTING

SEQ ID NO: 1 sets out the polynucleotide sequence from Alicyclobacillus pohliae NCIMB14276 encoding the wild type signal sequence (set out in nucleotides 1 to 99), an alpha-amylase polypeptide (set out in nucleotides 100 to 2157), and a stop codon at the 3′-terminus (set out in nucleotides 2157 to 2160).

SEQ ID NO: 2 sets out the amino acid sequence of the Alicyclobacillus pohliae NCIMB14276 wild type signal sequence (set out in amino acids 1 to 33) and an alpha-amylase polypeptide (set out in amino acids 34 to 719).

SEQ ID NO: 3 sets out a codon optimised polynucleotide sequence from Alicyclobacillus pohliae NCIMB14276 encoding the wild type signal sequence (set out in nucleotides 1 to 99), an alpha-amylase polypeptide (set out in nucleotides 100 to 2157), and a stop codon at the 3′-terminus (set out in nucleotides 2157 to 2160).

SEQ ID NO: 4 sets out the amino acid sequence of a G4-forming amylase from Pseudomonas saccharophila.

SEQ ID NO: 5 sets out the amino acid sequence of another G4-forming amylase.

### DETAILED DESCRIPTION OF THE INVENTION

Throughout the present specification and the accompanying claims the words “comprise” and “include” and variations such as “comprises”, “comprising”, “includes” and “including” are to be interpreted as open and inclusive. That is, these words are intended to convey the possible inclusion of other elements or integers not specifically recited, where the context allows.

Throughout the present specification and the accompanying claims the wording “nucleotides 100 to 2157” means nucleotides 100 up to and including 2157.

Throughout the present specification and the accompanying claims the wording “amino acids 34 to 719” means amino acids 34 up to and including 719.

The terms “polypeptide having an amino acid sequence as set out in amino acids 34 to 719 of SEQ ID NO: 2, “the mature polypeptide as set out in SEQ ID NO: 2” and “mature DSM-AM” and “mature alpha-amylase polypeptide” are used interchangeably herein.

The terms “according to the invention” and “of the invention” are used interchangeably herein.

In the context of the present invention “mature polypeptide” is defined herein as a polypeptide having alpha-amylase activity that is in its final form following translation and any post-translational modifications, including N-terminal processing, C-terminal truncation, glycosylation, phosphorylation, etc. The process of maturation may depend on the particular expression vector used, the expression host and the production process.

The terms reference alpha-amylase polypeptide and reference polypeptide having alpha-amylase activity are used interchangeably herein.

The reference alpha-amylase polypeptide, also referred to as reference polypeptide having alpha-amylase activity herein and herein after, is preferably the alpha-amylase as set out in amino acids 34 to 317 of SEQ ID NO: 2.

The term alpha-amylase polypeptide herein and herein after includes alpha-amylase polypeptide variants. An alpha-amylase polypeptide variant comprises at least one substitution at a position (in the variant) corresponding to one of the positions set out above in amino acids 34 to 719 as set out in SEQ ID NO: 2.

The present invention relates to an enzyme composition comprising an alpha-amylase polypeptide comprising

(a) an amino acid sequence as set out in amino acids 34 to 719 of SEQ ID NO: 2; or

(b) an amino acid sequence having at least 99.5% identity to an amino acid sequence as set out in amino acids 34 to 719 of SEQ ID NO: 2; or

(c) an amino acid sequence encoded by a polynucleotide as set out in nucleotides 100 to 2157 of SEQ ID NO: 1 or SEQ ID NO: 3; or

(d) an amino acid sequence having at least 70% identity to an amino acid sequence as set out in amino acids 34 to 719 of SEQ ID NO: 2 and having at least one of Asp at position 184, Ala at position 297, Thr at position 368 and Asn at position 489, said positions being defined with reference to SEQ ID NO: 2; or

(e) an amino acid sequence having at least 70% identity to an amino acid sequence as set out in amino acids 34 to 719 of SEQ ID NO: 2 and having at least one of Asp at position 184, Ala at position 297, Thr at position 368 and Asn at position 489, said positions being defined with reference to SEQ ID NO: 2 and said amino acid sequence characterized in that when used to prepare a baked product having a least 5 wt % sugar based on flour, said baked product has reduced hardness after storage in comparison with a baked product prepared without use of said amino acid sequence; or

(f) an amino acid sequence having alpha-amylase activity and having at least 70% identity to an amino acid sequence as set out in amino acids 34 to 719 of SEQ ID NO: 2 and having a substitution, at any one or more positions corresponding to 37, 39, 46, 47, 48, 49, 53, 78, 80, 84, 87, 94, 101, 102, 103, 104, 105, 106, 107, 108, 110, 111, 113, 115, 120, 121, 127, 128, 130, 133, 136, 137, 150, 157, 158, 159, 161, 162, 163, 166, 167, 169, 176, 177, 179, 201, 207, 210, 211, 216, 219, 221, 222, 223, 227, 228, 232, 233, 234, 237, 240, 243, 247, 250, 252, 255, 258, 260, 266, 267, 268, 269, 273, 284, 285, 287, 291, 292, 293, 295, 296, 297, 299, 300, 302, 304, 306, 312, 314, 315, 316, 317, 319, 321, 332, 355, 356, 358, 360, 361, 364, 367, 383, 389, 391, 400, 403, 404, 407, 410, 411, 421, 424, 447, 454, 455, 478, 483, 500, 521, 538, 569, 581, 616, 621, 636, 670, 681, 684, 685, 693, 709, 710,

said positions being defined with reference to an amino acid sequence as set out in amino acids 34 to 719 of SEQ ID NO: 2;
and wherein the composition further comprises a G4-forming amylase having an amino acid sequence at least 70% identical to the amino acid sequence as set out in SEQ ID NO: 4.

The enzyme composition according to the invention has a synergistic effect on an improved property, preferably a synergistic effect on the reduction of hardness after storage of a baked and/or to the reduction of loss of resilience over storage of a baked product.

In an embodiment of the invention, the alpha-amylase polypeptide has at least 70% identity with an amino acid sequence as set out in amino acids 34 to 317 of SEQ ID NO: 2,

and has a substitution at any one or more positions corresponding to

46, 48, 49, 53, 78, 94, 101, 103, 105, 108, 110, 111, 121, 127, 157, 159, 161, 162, 162, 166, 167, 169, 201, 207, 210, 211, 219, 221, 227, 228, 232, 233, 243, 252, 255, 258, 267, 287, 294, 297, 300, 302, 304, 314, 315, 316, 317, 321, 356, 358, 360, 364, 367, 391, 403, 404, 410, 421, 454, 483, 685,

said positions being defined with reference to an amino acid sequence as set out in amino acids 34 to 719 of SEQ ID NO: 2;

and wherein the alpha-amylase polypeptide preferably demonstrates any one of

increased thermostability, or

increased sucrose tolerance, or

increased Activity at pH4: Activity at pH 5 ratio

as compared with a reference polypeptide having an amino acid sequence as set out in amino acids 34 to 317 of SEQ ID NO: 2.

In an aspect of the invention, the alpha-amylase polypeptide variant has at least 70% identity with an amino acid sequence as set out in amino acids 34 to 317 of SEQ ID NO: 2,

and has a substitution at any one or more positions corresponding to

46, 94, 101, 103, 108, 121, 161, 166, 201, 221, 233, 255, 287, 294, 297, 314, 315, 360, 404, 421,

said positions being defined with reference to an amino acid sequence as set out in amino acids 34 to 719 of SEQ ID NO: 2;

and wherein the variant has an increased thermostability compared with a reference polypeptide as set out in amino acids 34 to 317 of SEQ ID NO: 2.

Sucrose Tolerance

Sucrose tolerance may be determined by measuring the activity in the presence of increasing concentration of sucrose (for example incubate with Phadebas tablets for 15 min at 60° C. in the presence of 0-40% (by weight) sucrose) expressed as a percentage of the activity at 0% sucrose. The activity may be determined using a suitable assay such as the NBAU assay or Maltotriose assay as described herein.

Sucrose tolerance of an alpha-amylase polypeptide variant may be expressed as the ratio of

[Activity of an alpha-amylase polypeptide in the presence of sucrose] to [Activity of the alpha-amylase polypeptide in the absence of sucrose],

expressed as a percentage of the ratio of

[Activity of a reference polypeptide having alpha-amylase activity in the presence of sucrose] to [Activity of the reference polypeptide having alpha-amylase activity in the absence of sucrose].

Sucrose tolerance of an alpha-amylase polypeptide variant may be expressed as the ratio of

[Activity on maltotriose of an alpha-amylase polypeptide in the presence of sucrose] to [Activity on maltotriose of the alpha-amylase polypeptide in the absence of sucrose],

expressed as a percentage of the ratio of

[Activity on maltotriose of a reference polypeptide having alpha-amylase activity in the presence of sucrose] to [Activity on maltotriose of the reference polypeptide having alpha-amylase activity in the absence of sucrose].

The percentage thus obtained may be used as measure for the sucrose tolerance of the alpha-amylase polypeptide variant. A sucrose tolerance of more than 100% shows that the alpha-amylase polypeptide variant has an increased sucrose tolerance compared to the reference polypeptide having alpha-amylase activity. In an aspect of the invention the alpha-amylase polypeptide variant has an increased sucrose tolerance compared with a reference polypeptide having alpha-amylase activity, wherein the reference polypeptide having alpha-amylase activity has an amino acid sequence as set out in amino acids 34 to 317 of SEQ ID NO: 2.

Activity at pH4: Activity to pH5 Ratio

Activity at pH 4 and Activity at pH 5 may be determined using a suitable assay such as the NBAU assay or Maltotriose assay as described herein and adjusting the pH accordingly.

Activity at pH4: Activity to pH5 ratio of an alpha-amylase polypeptide variant may be expressed as the ratio of

[Activity of an alpha-amylase polypeptide determined at pH 4] to [Activity of the alpha-amylase polypeptide determined at pH 5],

expressed as a percentage of the ratio of

[Activity of a reference polypeptide having alpha-amylase activity at pH 4] to [Activity of the reference polypeptide at a pH 5].

Activity at pH4: Activity to pH5 ratio of an alpha-amylase polypeptide variant according to the invention may be expressed as the ratio of

[Activity on maltotriose of a alpha-amylase polypeptide determined at pH 4] to [Activity on maltotriose of the alpha-amylase polypeptide determined at pH 5], expressed as a percentage of the ratio of

[Activity on maltotriose of a reference polypeptide having alpha-amylase activity at pH 4] to [Activity on maltotriose of the reference polypeptide at pH 5].

The percentage thus obtained may be used as measure for the Activity at pH4: Activity to pH5 ratio of the alpha-amylase polypeptide variant. An Activity at pH4: Activity to pH5 ratio of more than 100% shows that the alpha-amylase polypeptide variant has an increased Activity at pH4: Activity to pH5 ratio compared to the reference polypeptide having alpha-amylase activity that the alpha-amylase polypeptide has an increased Activity at pH4: Activity to pH5 ratio compared to the reference polypeptide having alpha-amylase activity.

In an aspect of the invention the alpha-amylase polypeptide variant has an increased Activity at pH4: Activity to pH5 ratio compared with a reference polypeptide having alpha-amylase activity, wherein the reference polypeptide having alpha-amylase activity has an amino acid sequence as set out in amino acids 34 to 317 of SEQ ID NO: 2.

Thermostability

Thermostability may be determined by measuring the residual activity after incubation at a higher temperature (e.g. 50-100° C. for 1-30 min), using a suitable assay such as the NBAU assay or Maltotriose assay as described herein.

Thermostability may be determined at a suitable pH such as at pH 4 or at pH 5. Thermostability may be determined in the presence of sucrose.

In an aspect of the invention the alpha-amylase polypeptide variant has an increased thermostability as compared with a reference polypeptide having alpha-amylase activity, wherein the reference polypeptide having alpha-amylase activity has an amino acid sequence as set out in amino acids 34 to 317 of SEQ ID NO: 2.

Thermostability at pH 5

Thermostability at pH 5 of an alpha-amylase polypeptide variant may be expressed as the ratio of

[Residual Activity of an alpha-amylase polypeptide determined after an incubation at a temperature of above 37 degrees Celsius at pH 5] to [Activity of the alpha-amylase polypeptide determined after an incubation at a temperature of 37 degrees Celsius at pH 5],

expressed as a percentage of the ratio of

[Residual Activity of a reference polypeptide having alpha-amylase activity after an incubation at a temperature of above 37 degrees Celsius at pH 5] to [Activity of the reference polypeptide having alpha-amylase activity after an incubation at a temperature of 37 degrees Celsius at pH 5].

Thermostability at pH 5 of an alpha-amylase polypeptide variant may be expressed as the ratio of

[Residual Activity on maltotriose of an alpha-amylase polypeptide determined after an incubation at a temperature of above 37 degrees Celsius at pH 5] to [Activity on maltotriose of the alpha-amylase polypeptide determined after an incubation at a temperature of 37 degrees Celsius at pH 5],

expressed as a percentage of the ratio of

[Residual Activity on maltotriose of a reference polypeptide having alpha-amylase activity after an incubation at a temperature of above 37 degrees Celsius at pH 5] to [Activity on maltotriose of the reference polypeptide having alpha-amylase activity after an incubation at a temperature of 37 degrees Celsius at pH 5].

The percentage thus obtained may be used as measure for the thermostability at pH 5 of the alpha-amylase polypeptide variant. A thermostability at pH 5 of more than 100% shows that the alpha-amylase polypeptide variant has an increased thermostability at pH 5 compared to the reference polypeptide having alpha-amylase activity.

In an aspect of the invention the alpha-amylase polypeptide variant has an increased thermostability at pH 5 compared with a reference polypeptide having alpha-amylase activity, wherein the reference polypeptide having alpha-amylase activity has an amino acid sequence as set out in amino acids 34 to 317 of SEQ ID NO: 2.

Thermostability at pH 4

Thermostability at pH 4 of an alpha-amylase polypeptide variant may be expressed as the ratio of

[Residual Activity of an alpha-amylase polypeptide determined after an incubation at a temperature of above 37 degrees Celsius at pH 4] to [Activity of the alpha-amylase polypeptide determined after an incubation at a temperature of 37 degrees Celsius at pH 4],

expressed as a percentage of the ratio of

[Activity of a reference polypeptide reference polypeptide having alpha-amylase activity after an incubation temperature of above 37 degrees Celsius at pH 4] to [Activity of the reference polypeptide after an incubation at a temperature of 37 degrees Celsius at pH 4].

Thermostability at pH 4 of an alpha-amylase polypeptide variant according to the invention may be expressed as the ratio of

[Residual Activity on maltotriose of an alpha-amylase polypeptide determined after an incubation at a temperature of above 37 degrees Celsius at pH 4] to [Activity on maltotriose of the alpha-amylase polypeptide determined after an incubation at a temperature of 37 degrees Celsius at pH 4],

expressed as a percentage of the ratio of

[Activity on maltotriose of a reference polypeptide reference polypeptide having alpha-amylase activity after an incubation temperature of above 37 degrees Celsius at pH 4] to [Activity on maltotriose of the reference polypeptide after an incubation at a temperature of 37 degrees Celsius at pH 4].

The percentage thus obtained may be used as measure for the thermostability at pH 4 of the alpha-amylase polypeptide variant. A thermostability at pH 4 of more than 100% shows that the alpha-amylase polypeptide variant has an increased thermostability at pH 4 compared to the reference polypeptide having alpha-amylase activity.

In an aspect of the invention the alpha-amylase polypeptide variant has an increased thermostability at pH 4 compared with a reference polypeptide having alpha-amylase activity, wherein the reference polypeptide having alpha-amylase activity has an amino acid sequence as set out in amino acids 34 to 317 of SEQ ID NO: 2.

Thermostability in the Presence of Sucrose

Thermostability in the presence of sucrose of an alpha-amylase polypeptide variant may be expressed as the ratio of

[Residual Activity of an alpha-amylase polypeptide variant determined after incubation in the presence of sucrose at a temperature of above 37 degrees Celsius] to [Activity of the alpha-amylase polypeptide variant determined after incubation in the absence of sucrose at a temperature of 37 degrees Celsius],

expressed as a percentage of the ratio of

[Residual Activity of a reference polypeptide having alpha-amylase activity determined after incubation in the presence of sucrose at a temperature of above 37 degrees Celsius] to [Activity of the reference polypeptide determined after incubation in the absence of sucrose at a temperature of 37 degrees Celsius].

Thermostability in the presence of sucrose of an alpha-amylase polypeptide variant may be expressed as the ratio of

[Residual Activity on maltotriose of an alpha-amylase polypeptide variant determined after incubation in the presence of sucrose at a temperature of above 37 degrees Celsius] to [Activity on maltotriose of the alpha-amylase polypeptide variant determined after incubation in the absence of sucrose at a temperature of 37 degrees Celsius],

expressed as a percentage of the ratio of

[Residual Activity on maltotriose of a reference polypeptide having alpha-amylase activity determined after incubation in the presence of sucrose at a temperature of above 37 degrees Celsius] to [Activity on maltotriose of the reference polypeptide determined after incubation in the absence of sucrose at a temperature of 37 degrees Celsius].

The percentage thus obtained may be used as measure for the thermostability in the presence of sucrose of the alpha-amylase polypeptide variant. A thermostability in the presence of sucrose of more than 100% shows that the alpha-amylase polypeptide variant has an increased thermostability in the presence of sucrose compared to the reference polypeptide having alpha-amylase activity.

In an aspect of the invention the alpha-amylase polypeptide variant according to the invention has an increased thermostability in the presence of sucrose compared with a reference polypeptide having alpha-amylase activity, wherein the reference polypeptide having alpha-amylase activity has an amino acid sequence as set out in amino acids 34 to 317 of SEQ ID NO: 2.

Polypeptides

The invention provides an enzyme composition comprising at least two (isolated) polypeptides having starch degrading activity, namely an alpha-amylase and G4-forming amylase.

The terms “peptide” and “oligopeptide” are considered synonymous (as is commonly recognized) and each term can be used interchangeably as the context requires to indicate a chain of at least two amino acids coupled by peptidyl linkages. The word “polypeptide” (or protein) is used herein for chains containing more than seven amino acid residues. All oligopeptide and polypeptide formulas or sequences herein are written from left to right and in the direction from amino terminus to carboxy terminus. The three-letter code of amino acids used herein is commonly known in the art and can be found in Sambrook, et al. (Molecular Cloning: A Laboratory Manual, 3^{rd} ed., Cold Spring Harbor Laboratory Press, Cold Spring Harbor, N. Y., 2001).

The one-letter code of amino acids used herein is also commonly known in the art and can be found in Stryer (Biochemistry, 3^{rd} edition, W.H. Freeman and company new York, 1975)

The invention provides an enzyme composition comprising an alpha-amylase polypeptide comprising

(a) an amino acid sequence as set out in amino acids 34 to 719 of SEQ ID NO: 2; or

(b) an amino acid sequence having at least 99.5% identity to an amino acid sequence as set out in amino acids 34 to 719 of SEQ ID NO: 2; or

(c) an amino acid sequence encoded by a polynucleotide as set out in nucleotides 100 to 2157 of SEQ ID NO: 1 or SEQ ID NO: 3; or

(d) an amino acid sequence having at least 70% identity to an amino acid sequence as set out in amino acids 34 to 719 of SEQ ID NO: 2 and having at least one of Asp at position 184, Ala at position 297, Thr at position 368 and Asn at position 489, said positions being defined with reference to SEQ ID NO: 2; or

(e) an amino acid sequence having at least 70% identity to an amino acid sequence as set out in amino acids 34 to 719 of SEQ ID NO: 2 and having at least one of Asp at position 184, Ala at position 297, Thr at position 368 and Asn at position 489, said positions being defined with reference to SEQ ID NO: 2 and said amino acid sequence characterized in that when used to prepare a baked product having a least 5 wt % sugar based on flour, said baked product has reduced hardness after storage in comparison with a baked product prepared without use of said amino acid sequence; or

(f) an amino acid sequence having alpha-amylase activity and having at least 70% identity to an amino acid sequence as set out in amino acids 34 to 719 of SEQ ID NO: 2 and having a substitution, at any one or more positions corresponding to 37, 39, 46, 47, 48, 49, 53, 78, 80, 84, 87, 94, 101, 102, 103, 104, 105, 106, 107, 108, 110, 111, 113, 115, 120, 121, 127, 128, 130, 133, 136, 137, 150, 157, 158, 159, 161, 162, 163, 166, 167, 169, 176, 177, 179, 201, 207, 210, 211, 216, 219, 221, 222, 223, 227, 228, 232, 233, 234, 237, 240, 243, 247, 250, 252, 255, 258, 260, 266, 267, 268, 269, 273, 284, 285, 287, 291, 292, 293, 295, 296, 297, 299, 300, 302, 304, 306, 312, 314, 315, 316, 317, 319, 321, 332, 355, 356, 358, 360, 361, 364, 367, 383, 389, 391, 400, 403, 404, 407, 410, 411, 421, 424, 447, 454, 455, 478, 483, 500, 521, 538, 569, 581, 616, 621, 636, 670, 681, 684, 685, 693, 709, 710,

said positions being defined with reference to an amino acid sequence as set out in amino acids 34 to 719 of SEQ ID NO: 2;
and wherein the composition further comprises a G4-forming amylase having an amino acid sequence at least 70% identical to the amino acid sequence as set out in SEQ ID NO: 4.

The one or more amino acids of the polypeptides described herein may be substituted in order to improve the expression in a host cell. One or more amino acids of the polypeptides described herein may be substituted to change the enzymes specific activity, including sugar tolerance or thermal stability.

Conservative amino acid substitutions refer to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulphur-containing side chains is cysteine and methionine.

Preferred conservative amino acids substitution groups include: valine-leucine-isoleucine, phenylalanine-tyrosine, lysine-arginine, alanine-valine, and asparagine-glutamine. Substitutional variants of the amino acid sequence disclosed herein are those in which at least one residue in the disclosed sequences has been removed and a different residue inserted in its place. Preferably, the amino acid change is conservative. Preferred conservative substitutions for each of the naturally occurring amino acids include: Ala to ser; Arg to lys; Asn to gln or his; Asp to glu; Cys to ser or ala; Gln to asn; Glu to asp; Gly to pro; His to asn or gln; He to leu or val; Leu to ile or val; Lys to arg; gln or glu; Met to leu or ile; Phe to met, leu or tyr; Ser to thr; Thr to ser; Trp to tyr; Tyr to trp or phe; and, Val to ile or leu.

Polypeptides described herein may be in a substantially isolated form. It will be understood that the polypeptide may be mixed with carriers or diluents which will not interfere with the intended purpose of the polypeptide and still be regarded as substantially isolated. The polypeptide of the invention may also be in a substantially purified form, in which case it will generally comprise the polypeptide in a preparation in which more than 50%. e.g. more than 80%, 90%, 95% or 99%, by weight of the polypeptide in the preparation is a polypeptide of the invention.

For example, recombinantly produced polypeptides and proteins produced in host cells are considered isolated for the purpose of the invention as are native or recombinant polypeptides which have been substantially purified by any suitable technique such as, for example, the single-step purification method disclosed in Smith and Johnson, Gene 67:31-40 (1988).

The polypeptides described herein may be chemically modified, e.g. post-translationally modified. For example, they may be glycosylated or comprise modified amino acid residues. They may also be modified by the addition of Histidine residues or a T7 tag to assist their purification or by the addition of a signal sequence to promote their secretion from a cell. Such modified polypeptides and proteins fall within the scope of the term “polypeptide” of the invention.

For secretion of the translated protein into the lumen of the endoplasmic reticulum, into the periplasmic space or into the extracellular environment, an appropriate secretion signal sequence may be fused to the polynucleotide of the invention. The signals may be endogenous to the polypeptide or they may be heterologous signals.

The polypeptides described herein may be produced in a modified form, such as a fusion protein, and may include not only secretion signals but also additional heterologous functional regions. Thus, for instance, a region of additional amino acids, particularly charged amino acids, may be added to the N-terminus of the polypeptide to improve stability and persistence in the host cell, during purification or during subsequent handling and storage. Also, peptide moieties may be added to the polypeptide to facilitate purification.

Polypeptides described herein include naturally purified products, products of chemical synthetic procedures, and products produced by recombinant techniques from a prokaryotic or eukaryotic host, including, for example, bacterial, yeast, higher plant, insect and mammalian cells. Depending upon the host employed in a recombinant production procedure, the polypeptides of the present invention may be glycosylated or may be non-glycosylated. In addition, polypeptides of the invention may also include an initial modified methionine residue, in some cases as a result of host-mediated processes.

Alpha-Amylase Polypeptide

The alpha-amylase polypeptide herein is a starch degrading enzyme. An alpha-amylase polypeptide variant will typically retain alpha-amylase activity. That is to say, the alpha-amylase polypeptide variant will typically be capable of alpha-amylase activity. Alpha-amylase activity can suitably be determined using the Ceralpha® procedure, which is recommended by the American Association of Cereal Chemists (AACC). Alpha-amylase activity may for example be determined using a Megazyme Ceralpha alpha-amylase assay kit (Megazyme International Ireland Ltd., Co. Wicklow, Ireland) according to the manufacturer's instruction.

The alpha-amylase polypeptide herein has an amino acid sequence having at least 70% identity to an amino acid sequence as set out in amino acids 34 to 719 of SEQ ID NO: 2.

In an aspect the alpha-amylase polypeptide herein has an amino acid sequence having at least 75% identity, in an aspect at least 80% identity, in an aspect at least 85% identity, in an aspect at least 90% identity, in an aspect at least 95% identity, in an aspect at least 99% identity to the amino acid sequence as set out in amino acids 34 to 719 of SEQ ID NO: 2.

In an aspect the alpha-amylase polypeptide herein has an amino acid sequence having at least at least 99.5% identity to the amino acid sequence as set out in as set out in amino acids 34 to 719 of SEQ ID NO: 2.

The alpha-amylase polypeptide herein comprises

(a) an amino acid sequence as set out in amino acids 34 to 719 of SEQ ID NO: 2; or

(b) an amino acid sequence having at least 99.5% identity to an amino acid sequence as set out in amino acids 34 to 719 of SEQ ID NO: 2; or

(c) an amino acid sequence encoded by a polynucleotide as set out in nucleotides 100 to 2157 of SEQ ID NO: 1 or SEQ ID NO: 3; or

(d) an amino acid sequence having at least 70% identity to an amino acid sequence as set out in amino acids 34 to 719 of SEQ ID NO: 2 and having at least one of Asp at position 184, Ala at position 297, Thr at position 368 and Asn at position 489, said positions being defined with reference to SEQ ID NO: 2; or

(e) an amino acid sequence having at least 70% identity to an amino acid sequence as set out in amino acids 34 to 719 of SEQ ID NO: 2 and having at least one of Asp at position 184, Ala at position 297, Thr at position 368 and Asn at position 489, said positions being defined with reference to SEQ ID NO: 2 and said amino acid sequence characterized in that when used to prepare a baked product having a least 5 wt % sugar based on flour, said baked product has reduced hardness after storage in comparison with a baked product prepared without use of said amino acid sequence; or

(f) an amino acid sequence having alpha-amylase activity and having at least 70% identity to an amino acid sequence as set out in amino acids 34 to 719 of SEQ ID NO: 2 and having a substitution, at any one or more positions corresponding to 37, 39, 46, 47, 48, 49, 53, 78, 80, 84, 87, 94, 101, 102, 103, 104, 105, 106, 107, 108, 110, 111, 113, 115, 120, 121, 127, 128, 130, 133, 136, 137, 150, 157, 158, 159, 161, 162, 163, 166, 167, 169, 176, 177, 179, 201, 207, 210, 211, 216, 219, 221, 222, 223, 227, 228, 232, 233, 234, 237, 240, 243, 247, 250, 252, 255, 258, 260, 266, 267, 268, 269, 273, 284, 285, 287, 291, 292, 293, 295, 296, 297, 299, 300, 302, 304, 306, 312, 314, 315, 316, 317, 319, 321, 332, 355, 356, 358, 360, 361, 364, 367, 383, 389, 391, 400, 403, 404, 407, 410, 411, 421, 424, 447, 454, 455, 478, 483, 500, 521, 538, 569, 581, 616, 621, 636, 670, 681, 684, 685, 693, 709, 710,

said positions being defined with reference to an amino acid sequence as set out in amino acids 34 to 719 of SEQ ID NO: 2,
and wherein the composition further comprises a G4-forming amylase having an amino acid sequence at least 70% identical to the amino acid sequence as set out in SEQ ID NO: 4.

In an embodiment the alpha-amylase polypeptide comprises the amino acid sequence having at least 99.5% identity, preferably at least 99.6% identity, preferably at least 99.7% identity preferably at least 99.8% identity, preferably at least 99.9% identity to a polypeptide having an amino acid sequence as set out in amino acids 34 to 719 of SEQ ID NO: 2 which has alpha-amylase activity. In general, the naturally occurring amino acid sequence shown in amino acids 34 to 719 of SEQ ID NO: 2 is preferred.

In an aspect the alpha-amylase polypeptide comprises an amino acid sequence having at least 70% identity, in an aspect at least 80% identity, in an aspect at least 85% identity, in an aspect at least 90% identity, in an aspect at least 95% identity to an amino acid sequence as set out in amino acids 34 to 719 of SEQ ID NO: 2 and having at least one of Asp at position 184, Ala at position 297, Thr at position 368 and Asn at position 489, said positions being defined with reference to SEQ ID NO: 2.

As is known to the person skilled in the art it is possible that the N- and/or C-termini of SEQ ID NO: 2 or of the mature alpha-amylase polypeptide in the amino acid sequence according to SEQ ID NO: 2 (as set out in amino acids 34 to 719) might be heterogeneous, due to variations in processing during maturation. In particular such processing variations might occur upon overexpression of the polypeptide. In addition, exo-protease activity might give rise to heterogeneity. The extent to which heterogeneity occurs depends also on the host and fermentation protocols that are used. Such C-terminal processing artefacts might lead to shorter polypeptides or longer polypeptides as indicated with SEQ ID NO: 2 or with the mature alpha-amylase polypeptide in the amino acid sequence according to SEQ ID NO: 2. As a result of such processing variations the N-terminus might also be heterogeneous. Processing variants at the N-terminus could be due to alternative cleavage of the signal sequence by signal peptidases.

The alpha-amylase polypeptide in an aspect has at least 99.5% sequence identity to the sequence set out in SEQ ID NO: 2.

The sequence of the polypeptide of SEQ ID NO: 2 can thus be modified to provide polypeptides of the invention. Amino acid substitutions may be made, for example, 1, 2, 3 or 4 substitutions. The modified polypeptide retains activity as an alpha amylase.

In an aspect the alpha-amylase polypeptide has at least 70% identity, in an aspect at least 80% identity, in an aspect at least 85% identity, in an aspect at least 90% identity, in an aspect at least 95% identity, in an aspect at least 99.5% identity to a polypeptide having an amino acid sequence as set out in SEQ ID NO: 2 or having an amino acid sequence as set out in amino acids 34 to 719 of SEQ ID NO: 2.

Preferably, such an polypeptide has an amino acid sequence which, when aligned with the amino acid sequence as set in SEQ ID NO 2, comprises at least one of Asp at position 184, Ala at position 297, Thr at position 368 and Asn at position 489, said positions being defined with reference to SEQ ID NO: 2. Preferably such an alpha-amylase comprises at least Ala at position 297 said position being defined with reference to SEQ ID NO: 2.

In an aspect the alpha-amylase polypeptide may comprise at least two of Asp at position 184, Ala at position 297, Thr at position 368 and Asn at position 489, said positions being defined with reference to SEQ ID NO: 2. Preferably such a polypeptide comprises at least: Asp at position 184 and Ala at position 297; at least Ala at position 297 and Thr at position 368; or at least Ala at position 297 and Asn at position 489, all of said positions being defined with reference to SEQ ID NO: 2.

In an aspect the alpha-amylase polypeptide may comprise at least three of Asp at position 184, Ala at position 297, Thr at position 368 and Asn at position 489, said positions being defined with reference to SEQ ID NO: 2. Preferably, such a polypeptide comprises at least: Ala at position 297, Thr at position 368 and Asn at position 489; Asp at position 184, Ala at position 297 and Thr at position 368; or Asp at position 184, Ala at position 297 and Asn at position 489, all of said positions being defined with reference to SEQ ID NO: 2.

In an aspect the alpha-amylase polypeptide may comprise Asp at position 184, Ala at position 297, Thr at position 368 and Asn at position 489, all of said positions being defined with reference to SEQ ID NO: 2.

In an aspect the alpha-amylase polypeptide comprises an amino acid sequence having at least 80% identity to an amino acid sequence as set out in amino acids 34 to 719 of SEQ ID NO: 2 and having at least one of Asp at position 184, Ala at position 297, Thr at position 368 and Asn at position 489, said positions being defined with reference to SEQ ID NO: 2.

In an aspect the alpha-amylase polypeptide comprises an amino acid sequence having at least 85% identity to an amino acid sequence as set out in amino acids 34 to 719 of SEQ ID NO: 2 and having at least one of Asp at position 184, Ala at position 297, Thr at position 368 and Asn at position 489, said positions being defined with reference to SEQ ID NO: 2.

In an aspect the alpha-amylase polypeptide comprises an amino acid sequence having at least 90% identity to an amino acid sequence as set out in amino acids 34 to 719 of SEQ ID NO: 2 and having at least one of Asp at position 184, Ala at position 297, Thr at position 368 and Asn at position 489, said positions being defined with reference to SEQ ID NO: 2.

In an aspect the alpha-amylase polypeptide comprises an amino acid sequence having at least 95% identity to an amino acid sequence as set out in amino acids 34 to 719 of SEQ ID NO: 2 and having at least one of Asp at position 184, Ala at position 297, Thr at position 368 and Asn at position 489, said positions being defined with reference to SEQ ID NO: 2.

In an aspect the alpha-amylase polypeptide comprises an amino acid sequence having at least 80% identity to an amino acid sequence as set out in amino acids 34 to 719 of SEQ ID NO: 2 and having Asp at position 184, Ala at position 297, Thr at position 368 and Asn at position 489, said positions being defined with reference to SEQ ID NO: 2.

In an aspect the alpha-amylase polypeptide comprises an amino acid sequence having at least 85% identity to an amino acid sequence as set out in amino acids 34 to 719 of SEQ ID NO: 2 and having Asp at position 184, Ala at position 297, Thr at position 368 and Asn at position 489, said positions being defined with reference to SEQ ID NO: 2.

In an aspect the alpha-amylase polypeptide comprises an amino acid sequence having at least 90% identity to an amino acid sequence as set out in amino acids 34 to 719 of SEQ ID NO: 2 and having Asp at position 184, Ala at position 297, Thr at position 368 and Asn at position 489, said positions being defined with reference to SEQ ID NO: 2.

In an aspect the alpha-amylase polypeptide comprises an amino acid sequence having at least 95% identity to an amino acid sequence as set out in amino acids 34 to 719 of SEQ ID NO: 2 and having Asp at position 184, Ala at position 297, Thr at position 368 and Asn at position 489, said positions being defined with reference to SEQ ID NO: 2.

In an aspect the alpha-amylase polypeptide comprises an amino acid sequence having at least 99.5% identity to an amino acid sequence as set out in amino acids 34 to 719 of SEQ ID NO: 2 and having Asp at position 184, Ala at position 297, Thr at position 368 and Asn at position 489, said positions being defined with reference to SEQ ID NO: 2.

In an aspect the alpha-amylase polypeptide comprises an amino acid sequence having at least 70% identity to an amino acid sequence as set out in amino acids 34 to 719 of SEQ ID NO: 2 and having at least one of Asp at position 184, Ala at position 297, Thr at position 368 and Asn at position 489, said positions being defined with reference to SEQ ID NO: 2 and said amino acid sequence characterized in that when used to prepare a baked product having a least 5 wt % sugar based on flour, said baked product has reduced hardness after storage in comparison with a baked product prepared without use of said amino acid sequence.

Embodiments of the alpha-amylase polypeptide include without any limitation polypeptides having alpha-amylase activity and having at least 70% identity to an amino acid sequence as set out in amino acids 34 to 719 of SEQ ID NO: 2 and having a substitution, at any one or more positions corresponding to

34, 35, 36, 37, 38, 39, 40, 41, 42, 43, 44, 45, 46, 47, 48, 49, 50, 60, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 251, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 351, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 451, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 551, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 651, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, said positions being defined with reference to an amino acid sequence as set out in amino acids 34 to 719 of SEQ ID NO: 2.

In an aspect the alpha-amylase polypeptide includes without any limitation polypeptides having alpha-amylase activity and having at least 70% identity to an amino acid sequence as set out in amino acids 34 to 719 of SEQ ID NO: 2 and having a substitution, at any one or more positions corresponding to

34, 35, 40, 45, 73, 74, 76, 77, 79, 86, 95, 99, 100, 103, 119, 120, 131, 141, 142, 148, 152, 163, 169, 171, 172, 174, 176, 187, 188, 192, 201, 203, 220, 234, 236, 247, 249, 261, 266, 268, 272, 275, 276, 280, 281, 285, 287, 297, 299, 305, 316, 320, 321, 327, 341, 342, 348, 365, 371, 375, 378, 397, 381, 389, 401, 403, 425, 436, 442, 454, 468, 474, 479, 483, 486, 487, 493, 494, 495, 496, 497, 498, 500, 507, 510, 513, 520, 526, 555, 564, 573, 575, 581, 583, 586, 589, 595, 618, 621, 624, 629, 636, 645, 664 and/or 681, said positions being defined with reference to an amino acid sequence as set out in amino acids 34 to 719 of SEQ ID NO: 2.

In an aspect the alpha-amylase polypeptide includes without any limitation polypeptides having alpha-amylase activity and having at least 70% identity, in an aspect at least 80% identity, in an aspect at least 85% identity, in an aspect at least 90% identity, in an aspect at least 95% identity to an amino acid sequence as set out in amino acids 34 to 719 of SEQ ID NO: 2 and having a substitution, at any one or more positions corresponding to

37, 39, 46, 47, 48, 49, 53, 78, 80, 84, 87, 94, 101, 102, 103, 104, 105, 106, 107, 108, 110, 111, 113, 115, 120, 121, 127, 128, 130, 133, 136, 137, 150, 157, 158, 159, 161, 162, 163, 166, 167, 169, 176, 177, 179, 201, 207, 210, 211, 216, 219, 221, 222, 223, 227, 228, 232, 233, 234, 237, 240, 243, 247, 250, 252, 255, 258, 260, 266, 267, 268, 269, 273, 284, 285, 287, 291, 292, 293, 295, 296, 297, 299, 300, 302, 304, 306, 312, 314, 315, 316, 317, 319, 321, 332, 355, 356, 358, 360, 361, 364, 367, 383, 389, 391, 400, 403, 404, 407, 410, 411, 421, 424, 447, 454, 455, 478, 483, 500, 521, 538, 569, 581, 616, 621, 636, 670, 681, 684, 685, 693, 709, 710,

said positions being defined with reference to an amino acid sequence as set out in amino acids 34 to 719 of SEQ ID NO: 2.

In an aspect the alpha-amylase polypeptide includes without any limitation polypeptides having alpha-amylase activity and having at least 70% identity to an amino acid sequence as set out in amino acids 34 to 719 of SEQ ID NO: 2 and having any one or more substitutions corresponding to F221L, D321G, T294P, the positions being defined with reference to SEQ ID NO:2.

That is to say, when the alpha-amylase polypeptide sequence is aligned with the sequence of the amino acids 34 to 719 of SEQ ID NO: 2, the variant will comprise at least one substitution at a position (in the variant) corresponding to one of the positions set out above in amino acids 34 to 719 as set out in SEQ ID NO: 2. A “substitution” in this context indicates that a position in the variant which corresponds to one of the positions set out above in SEQ ID NO: 2 comprises an amino acid residue which does not appear at that position in SEQ ID NO: 2.

The alpha-amylase polypeptide variant may comprise a substitution at one or more of the said positions, for example at two, three, four, at least 5, at least 10, at least 15, at least 20, at least 25, at least 30, at least 35, at least 40, at least 45 or at least 50 or at all of the said positions.

The alpha-amylase polypeptide variant may comprise one or more substitutions as defined herein. A “substitution” in this context indicates that a position in the variant which corresponds to one of the positions set out above in SEQ ID NO: 2 comprises an amino acid residue which does not appear at that position in the parent alpha-amylase polypeptide (the parent alpha-amylase polypeptide is preferably the polypeptide as set out in amino acids 34 to 317 of SEQ ID NO: 2).

The alpha-amylase polypeptide variant may be generated using any combination of substitutions as described herein.

In an aspect of the invention the alpha-amylase polypeptide variant has an amino acid sequence which, when aligned with the alpha-amylase comprising the sequence set out in amino acids 34 to 317 of SEQ ID NO: 2, comprises a substitution of the amino acid residue 166, said position being defined with reference to the polypeptide as set out in amino acids 34 to 317 of SEQ ID NO: 2.

In an aspect of the invention the alpha-amylase polypeptide variant has an amino acid sequence which, when aligned with the alpha-amylase comprising the sequence set out in amino acids 34 to 317 of SEQ ID NO: 2, comprises a substitution of the amino acid residue 166, said position being defined with reference to the polypeptide as set out in amino acids 34 to 317 of SEQ ID NO: 2, wherein the substitution is S166L.

In an aspect of the invention the alpha-amylase polypeptide variant has an amino acid sequence which, when aligned with the alpha-amylase comprising the sequence set out in amino acids 34 to 317 of SEQ ID NO: 2, comprises a substitution of the amino acid residue 587, said position being defined with reference to the polypeptide as set out in amino acids 34 to 317 of SEQ ID NO: 2.

In an aspect of the invention the alpha-amylase polypeptide variant has an amino acid sequence which, when aligned with the alpha-amylase comprising the sequence set out in amino acids 34 to 317 of SEQ ID NO: 2, comprises a substitution of the amino acid residue 587, said position being defined with reference to the polypeptide as set out in amino acids 34 to 317 of SEQ ID NO: 2, wherein the substitution is A587G.

An alpha-amylase polypeptide variant may also comprise additional modifications in comparison to the reference alpha-amylase polypeptide at positions other than those specified herein, for example, one or more additional substitutions, additions or deletions. A alpha-amylase polypeptide variant may comprise a combination of different types of modification of this sort. A alpha-amylase polypeptide variant may comprise one, two, three, four, least 5, at least 10, at least 15, at least 20, at least 25, at least 30 or more such modifications (which may all be of the same type or may be different types of modification). Typically, the additional modifications may be substitutions.

The alpha-amylase polypeptide variant herein has an amino acid sequence having at least 70% identity to an amino acid sequence as set out in amino acids 34 to 719 of SEQ ID NO: 2.

In an aspect the alpha-amylase polypeptide variant herein has an amino acid sequence having at least 75% identity, in an aspect at least 80% identity, in an aspect at least 85% identity, in an aspect at least 90% identity, in an aspect at least 95% identity, in an aspect at least 99% identity to the amino acid sequence as set out in amino acids 34 to 719 of SEQ ID NO: 2.

In an aspect the alpha-amylase polypeptide variant herein has an amino acid sequence having at least at least 99.5% identity to the amino acid sequence as set out in as set out in amino acids 34 to 719 of SEQ ID NO: 2.

In an aspect of the invention, the alpha-amylase polypeptide variant has at least 70% identity with an amino acid sequence as set out in amino acids 34 to 317 of SEQ ID NO: 2,

and has a substitution at any one or more positions corresponding to

46, 94, 101, 103, 108, 121, 161, 166, 201, 221, 233, 255, 287, 294, 297, 314, 315, 360, 404, 421,

said positions being defined with reference to an amino acid sequence as set out in amino acids 34 to 719 of SEQ ID NO: 2;

and wherein the variant has an increased thermostability compared with a reference polypeptide as set out in amino acids 34 to 317 of SEQ ID NO: 2.

In an aspect of the invention, the alpha-amylase polypeptide variant has an increased thermostability compared with the polypeptide as set out in amino acids 34 to 317 of SEQ ID NO: 2 and has at least 75%, in an aspect at least 80%, in an aspect at least 85%, in an aspect at least 90%, in an aspect at least 95%, in an aspect at least 96%, in an aspect at least 97% in an aspect at least 98%, in an aspect at least 99%, in an aspect at least 99.5% identity to an amino acid sequence as set out in amino acids 34 to 317 of SEQ ID NO: 2.

In an aspect of the invention, the alpha-amylase polypeptide variant has at least 70% identity with an amino acid sequence as set out in amino acids 34 to 317 of SEQ ID NO: 2,

and has at least one substitution of an amino acid residue corresponding to

Q46E, L94F, T101A, W103Y, L108F, G121A, F161I, S166T, F201Y, F221I, S233N, A255V, V287F, D294G, A297S, V314L, L315F, L315M, L315I, L315T, N360S, N404G, A421L,

said positions being defined with reference to an amino acid sequence as set out in amino acids 34 to 719 of SEQ ID NO: 2;

and wherein the variant has an increased thermostability compared with a reference polypeptide as set out in amino acids 34 to 317 of SEQ ID NO: 2.

In an aspect of the invention, the alpha-amylase polypeptide variant has at least 70% identity with an amino acid sequence as set out in amino acids 34 to 317 of SEQ ID NO: 2,

and has a substitution at any one or more positions corresponding to

94, 108, 121, 166, 201, 221, 233, 255, 287, 297, 314, 360, 46, 103, 161, 315, 421, 294,

said positions being defined with reference to an amino acid sequence as set out in amino acids 34 to 719 of SEQ ID NO: 2;

and wherein the variant has an increased thermostability at pH 5 compared with a reference polypeptide as set out in amino acids 34 to 317 of SEQ ID NO: 2.

In an aspect of the invention, the alpha-amylase polypeptide variant has an increased thermostability at pH 5 compared with the polypeptide as set out in amino acids 34 to 317 of SEQ ID NO: 2 and has at least 75%, in an aspect at least 80%, in an aspect at least 85%, in an aspect at least 90%, in an aspect at least 95%, in an aspect at least 96%, in an aspect at least 97% in an aspect at least 98%, in an aspect at least 99%, in an aspect at least 99.5% identity to an amino acid sequence as set out in amino acids 34 to 317 of SEQ ID NO: 2.

In an aspect of the invention, the alpha-amylase polypeptide variant has at least 70% identity with an amino acid sequence as set out in amino acids 34 to 317 of SEQ ID NO: 2,

and has at least one substitution of an amino acid residue corresponding to

L94F, L108F, G121A, S166T, F201Y, F221I, S233N, A255V, V287F, A297S, V314L, N360S, Q46E, W103Y, F161I, L315F, L315M, L315M, A421L, D294G,

said positions being defined with reference to an amino acid sequence as set out in amino acids 34 to 719 of SEQ ID NO: 2;

and wherein the variant has an increased thermostability at pH 5 compared with a reference polypeptide as set out in amino acids 34 to 317 of SEQ ID NO: 2.

In an aspect of the invention, the alpha-amylase polypeptide variant has at least 70% identity with an amino acid sequence as set out in amino acids 34 to 317 of SEQ ID NO: 2,

and has a substitution at any one or more positions corresponding to

121, 221, 233, 255, 103, 315, 315, 315, 315, 315,

said positions being defined with reference to an amino acid sequence as set out in amino acids 34 to 719 of SEQ ID NO: 2;

and wherein the variant has an increased thermostability at pH 4 compared with a reference polypeptide as set out in amino acids 34 to 317 of SEQ ID NO: 2.

In an aspect of the invention the alpha-amylase polypeptide variant has an increased thermostability at pH 4 compared with the polypeptide as set out in amino acids 34 to 317 of SEQ ID NO: 2 and has at least 75%, in an aspect at least 80%, in an aspect at least 85%, in an aspect at least 90%, in an aspect at least 95%, in an aspect at least 96%, in an aspect at least 97% in an aspect at least 98%, in an aspect at least 99%, in an aspect at least 99.5% identity to an amino acid sequence as set out in amino acids 34 to 317 of SEQ ID NO: 2.

In an aspect of the invention, the alpha-amylase polypeptide variant has at least 70% identity with an amino acid sequence as set out in amino acids 34 to 317 of SEQ ID NO: 2,

and has at least one substitution of an amino acid residue corresponding to

G121A, F221I, S233N, A255V, W103Y, L315F, L315I, L315M, L315T, L315M,

said positions being defined with reference to an amino acid sequence as set out in amino acids 34 to 719 of SEQ ID NO: 2;

and wherein the variant has an increased thermostability at pH 4 compared with a reference polypeptide as set out in amino acids 34 to 317 of SEQ ID NO: 2.

In an aspect of the invention, the alpha-amylase polypeptide variant has at least 70% identity with an amino acid sequence as set out in amino acids 34 to 317 of SEQ ID NO: 2,

and has a substitution at any one or more positions corresponding to

94, 108, 166, 201, 221, 233, 287, 297, 314, 360, 404, 101, 103, 315, 421, 294, said positions being defined with reference to an amino acid sequence as set out in amino acids 34 to 719 of SEQ ID NO: 2;

and wherein the variant has an increased thermostability in the presence of sucrose compared with a reference polypeptide as set out in amino acids 34 to 317 of SEQ ID NO: 2.

In an aspect of the invention, the alpha-amylase polypeptide variant has an increased thermostability in the presence of sucrose compared with the polypeptide as set out in amino acids 34 to 317 of SEQ ID NO: 2 and has at least 75%, in an aspect at least 80%, in an aspect at least 85%, in an aspect at least 90%, in an aspect at least 95%, in an aspect at least 96%, in an aspect at least 97% in an aspect at least 98%, in an aspect at least 99%, in an aspect at least 99.5% identity to an amino acid sequence as set out in amino acids 34 to 317 of SEQ ID NO: 2.

In an aspect of the invention, the alpha-amylase polypeptide variant has at least 70% identity with an amino acid sequence as set out in amino acids 34 to 317 of SEQ ID NO: 2,

and has at least one substitution of an amino acid residue corresponding to

L94F, L108F, S166T, F201Y, F221I, S233N, V287F, A297S, V314L, N360S, N404G, T101A, W103Y, L315F, L315I, L315M, L315T, L315M, A421L, D294G,

said positions being defined with reference to an amino acid sequence as set out in amino acids 34 to 719 of SEQ ID NO: 2;

and wherein the variant has an increased thermostability in the presence of sucrose compared with a reference polypeptide as set out in amino acids 34 to 317 of SEQ ID NO: 2.

In an aspect of the invention, the alpha-amylase polypeptide variant has at least 70% identity with an amino acid sequence as set out in amino acids 34 to 317 of SEQ ID NO: 2,

and has a substitution at any one or more positions corresponding to

48, 49, 78, 108, 127, 127, 162, 167, 207, 210, 211, 219, 227, 243, 267, 287, 314, 356, 358, 391, 404, 685, 46, 103, 105, 315, 315, 315, 316, 316, 317, 294, 110, 121, 111, 167, 166,

said positions being defined with reference to an amino acid sequence as set out in amino acids 34 to 719 of SEQ ID NO: 2;

and wherein the variant has an increased sucrose tolerance compared with a reference polypeptide as set out in amino acids 34 to 317 of SEQ ID NO: 2.

In an aspect of the invention, the alpha-amylase polypeptide variant has an increased sucrose tolerance compared with the polypeptide as set out in amino acids 34 to 317 of SEQ ID NO: 2 and has at least 75%, in an aspect at least 80%, in an aspect at least 85%, in an aspect at least 90%, in an aspect at least 95%, in an aspect at least 96%, in an aspect at least 97% in an aspect at least 98%, in an aspect at least 99%, in an aspect at least 99.5% identity to an amino acid sequence as set out in amino acids 34 to 317 of SEQ ID NO: 2.

In an aspect of the invention, the alpha-amylase polypeptide variant has at least 70% identity with an amino acid sequence as set out in amino acids 34 to 317 of SEQ ID NO: 2,

and has at least one substitution of an amino acid residue corresponding to

I48V, 149T, M78L, L108V, T127A, T127P, V162A, T167S, 1207L, W210F, D211N, K219Q, F227Y, L243F, N267P, V287F, V341L, T356N, 1358F, S391A, N404G, F685I, Q46E, W103Y, S105T, L315F, L315M, L315T, D316N, D316S, F317W, D294G, N110C, N121C, L111C, T167C, S166L,

said positions being defined with reference to an amino acid sequence as set out

in amino acids 34 to 719 of SEQ ID NO: 2;

and wherein the variant has an increased sucrose tolerance compared with a reference polypeptide as set out in amino acids 34 to 317 of SEQ ID NO: 2.

In an aspect of the invention, the alpha-amylase polypeptide variant has at least 70% identity with an amino acid sequence as set out in amino acids 34 to 317 of SEQ ID NO: 2,

and has a Cysteine amino acid at both positions 110 and 121 or a Cysteine amino acid at both positions 111 and 167, said positions being defined with reference to an amino acid sequence as set out in amino acids 34 to 719 of SEQ ID NO: 2;

and wherein the variant has an increased sucrose tolerance compared with a reference polypeptide as set out in amino acids 34 to 317 of SEQ ID NO: 2.

In an aspect of the invention, the alpha-amylase polypeptide variant has at least 70% identity with an amino acid sequence as set out in amino acids 34 to 317 of SEQ ID NO: 2,

and has a substitution at any one or more positions corresponding to

157, 159, 162, 169, 201, 219, 228, 232, 252, 255, 300, 302, 304, 321, 358, 364, 403, 410, 454, 483, 685, 53, 101, 105, 258, 315, 367,

said positions being defined with reference to an amino acid sequence as set out in amino acids 34 to 719 of SEQ ID NO: 2;

and wherein the variant has an increased Activity at pH4: Activity to pH5 ratio compared with a reference polypeptide as set out in amino acids 34 to 317 of SEQ ID NO: 2.

In an aspect of the invention, the alpha-amylase polypeptide variant has an increased Activity at pH4: Activity to pH5 ratio compared with the polypeptide as set out in amino acids 34 to 317 of SEQ ID NO: 2 and has at least 75%, in an aspect at least 80%, in an aspect at least 85%, in an aspect at least 90%, in an aspect at least 95%, in an aspect at least 96%, in an aspect at least 97% in an aspect at least 98%, in an aspect at least 99%, in an aspect at least 99.5% identity to an amino acid sequence as set out in amino acids 34 to 317 of SEQ ID NO: 2.

In an aspect of the invention, the alpha-amylase polypeptide variant has at least 70% identity with an amino acid sequence as set out in amino acids 34 to 317 of SEQ ID NO: 2,

and has at least one substitution of an amino acid residue corresponding to

V157I, V159I, V1262A, F169A, F201Y, K219Q, S228A, L232F, A252D, A255V, A255I, H300N, E302D, V304T, T321S, T321N, 1358F, S364D, G403N, G410A, 1454V, T483S, F685I, Y53L, Y53V, T101A, T101G, S105T, L258F, L315I, L315M, L367H,

said positions being defined with reference to an amino acid sequence as set out in amino acids 34 to 719 of SEQ ID NO: 2;

and wherein the variant has an increased Activity at pH4: Activity to pH5 ratio compared with a reference polypeptide as set out in amino acids 34 to 317 of SEQ ID NO: 2.

A nucleic acid molecule for the production of the alpha-amylase polypeptide having one or more substitutions (i.e. a variant) described herein can be generated using standard molecular biology techniques well known to those skilled in the art taken in combination with the sequence information provided herein.

For example, using standard synthetic techniques, the required nucleic acid molecule may be synthesized de novo. Such a synthetic process will typically be an automated process.

Alternatively, a nucleic acid molecule for the production of an alpha-amylase polypeptide as described herein, may be generated by use of site-directed mutagenesis of an existing nucleic acid molecule, for example a wild-type nucleic acid molecule. Site-directed mutagenesis may be carried out using a number of techniques well know to those skilled in the art.

In one such method, mentioned here merely by way of example, PCR is carried out on a plasmid template using oligonucleotide “primers” encoding the desired substitution. As the primers are the ends of newly-synthesized strands, should there be a mis-match during the first cycle in binding the template DNA strand, after that first round, the primer-based strand (containing the mutation) would be at equal concentration to the original template. After successive cycles, it would exponentially grow, and after 25, would outnumber the original, unmutated strand in the region of 8 million: 1, resulting in a nearly homogeneous solution of mutated amplified fragments. The template DNA may then be eliminated by enzymatic digestion with, for example using a restriction enzyme which cleaves only methylated DNA, such as Dpn1. The template, which is derived from an alkaline lysis plasmid preparation and therefore is methylated, is destroyed in this step, but the mutated plasmid is preserved because it was generated in vitro and is unmethylated as a result.

In such a method more than one mutation (encoding a substitution as described herein) may be introduced into a nucleic acid molecule in a single PCR reaction, for example by using one or more oligonucleotides, each comprising one or more mis-matches. Alternatively, more than one mutation may be introduced into a nucleic acid molecule by carrying out more than one PCR reaction, each reaction introducing one or more mutations, so that altered nucleic acids are introduced into the nucleic acid in a sequential, iterative fashion.

A nucleic acid for the production of an alpha-amylase polypeptide as described herein can be generated using cDNA, mRNA or alternatively, genomic DNA, as a template and appropriate mis-matched oligonucleotide primers according to the site-directed mutagenesis technique described above. A nucleic acid molecule derived in this way can be cloned into an appropriate vector and characterized by DNA sequence analysis.

### G4-Forming Amylase

The G4-forming amylase herein preferably has an amino acid sequence at least 70% identical to the amino acid sequence as set out in SEQ ID NO: 4.

Pseudomonas saccharophila expresses a maltotetraose-forming maltotetraohydrolase (EC 3.2.1.60) which may also be referred to as a G4-forming amylase herein and herein after. The nucleotide sequence of the P. saccharophila gene encoding the G4-forming amylase has been determined. Zhou et al., “Nucleotide sequence of the maltotetraohydrolase gene from Pseudomonas saccharophila,” FEBS Lett. 255: 37-41 (1989); GenBank Ace. No. X16732.

Suitable G4-forming amylases or variants thereof may include G4-forming amylases, also referred to as maltotetraohydrolases, as described in any one of WO9950399, WO2005007818, WO2004111217, WO2005003339, WO2005007818, WO2005007867, WO2006003461, WO2007007053, WO2007148224, WO2009083592, WO2009088465, WO2010133644, WO2010118269, or WO2010132157.

Suitable G4-forming amylases may include a G4-forming amylase having an amino acid sequence at least 70% identical to the amino acid sequence as set out in SEQ ID NO: 4 and having a substitution at any one of positions

7, 8, 32, 38, 49, 62, 63, 64, 67, 72, 73, 74, 75, 76, 104, 106, 107, 110, 112, 116, 119, 122, 123, 124, 125, 126, 128, 130, 137, 138, 140, 142, 143, 144, 148, 149, 150, 151, 154, 156, 163, 164, 168, 169, 182, 183, 192, 195, 196, 200, 202, 208, 213, 220, 222, 225, 226, 227, 232, 233, 234, 236, 237, 239, 253, 255, 257, 260, 264, 267, 269, 271, 276, 282, 285, 295, 297, 300, 302, 305, 308, 312, 323, 324, 325, 341, 358, 367, 379, 390, said positions being defined with reference to SEQ ID NO:4.

Suitable G4-forming amylases may include a G4-forming amylase having an amino acid sequence at least 70% identical to the amino acid sequence as set out in SEQ ID NO:4 and having a substitution at any one of positions

121, 161, 223, 146, 157, 158, 198, 229, 303, 306, 309, 316, 353, 26, 70, 145, 188, 272, 339 said positions being defined with reference to SEQ ID NO:4.

Suitable G4-forming amylases may include a G4-forming amylase having an amino acid sequence at least 70% identical to the amino acid sequence as set out in SEQ ID NO:4 and having a substitution at any one of positions

3, 33, 34, 70, 121, 134, 141, 146, 157, 161, 178, 179, 229, 307, 309, 334 said positions being defined with reference to SEQ ID NO:4.

Suitable G4-forming amylases may include a G4-forming amylase having an amino acid sequence at least 70% identical to the amino acid sequence as set out in SEQ ID NO:4 and having a substitution at any one of positions

134, 141, 157, 223, 307, 334, said positions being defined with reference to SEQ ID NO:4.

Suitable G4-forming amylases may include a G4-forming amylase having an amino acid sequence at least 70% identical to the amino acid sequence as set out in SEQ ID NO:4 and having a substitution at any one of positions 121 and 223, preferably G121D and/or G223A said positions being defined with reference to SEQ ID NO:4. The position 223 substitution may also comprise G223L. In an aspect G4-forming amylases may include a G4-forming amylase having an amino acid sequence at least 70% identical to the amino acid sequence as set out in SEQ ID NO:4 and has a substitution at positions 33, preferably N33, more preferably N33Y, 34, preferably D34, more preferably D34N, 178 and a substitution at position 179.

A suitable G4-forming amylase may include SEQ ID NO:18 of WO2009061380.

A suitable G4-forming amylase may include a polypeptide having amino acid sequence as set out in of SEQ ID NO: 4. An embodiment of a G4-forming amylase may include a polypeptide having an amino acid sequence as set out in SEQ ID NO: 5 as disclosed herein. An embodiment of a G4-forming amylase may include the polypeptide having an amino acid sequence as set out in SEQ ID NO: 18 as disclosed in WO2009061380.

A G4-forming amylase is an enzyme that is inter alia capable of catalysing the degradation of starch. In particular it is capable of cleaving α-D-(I->4) 0-glycosidic linkages in starch. It may be referred to as a glucan 1,4-alpha-maltotetraohydrolase (EC 3.2.1.60). It may also be referred as a maltotetraohydrolase.

Pseudomonas saccharophila (GenBank Acc. No. X16732) expresses a G4-forming amylase.

The G4-forming amylase may be a G-4 forming amylase as expressed by Pseudomonas saccharophila, the polypeptide as set out in SEQ ID NO:4 or a variant thereof. The G-4 forming amylase is capable of producing maltotetraose from either liquefied starch or other source of maltodextrins at a high temperature e.g. about 60° C. to about 75° C.

As used herein the term starch refers to any material comprised of the complex polysaccharide carbohydrates of plants such as corn, comprised of amylose and amylopectin.

The amylase with G4-forming activity was dosed in the examples described herein at a level to achieve an appropriate effect in baking. Suitable assays to determine the activity of the G4-forming amylase include assays known in the art such as Betamyl assay (Megazyme); Phadebas assay (Pharmacia & Upjohn Diagnostics AB). The NBAU assay as described herein may also be applied (Ceralpha, Megazyme as described herein).

In an aspect the G4-forming amylase has an amino acid sequence having at least 75% identity, in an aspect at least 80% identity, in an aspect at least 85% identity, in an aspect at least 90% identity, in an aspect at least 95% identity, in an aspect at least 99% identity to the amino acid sequence as set out in SEQ ID NO: 4.

### Sequence Identity

The terms “homology”, “percent identity”, “percent homology” and “percentage of identity” are used interchangeably herein. For the purpose of this invention, it is defined here that in order to determine the percent homology of two amino acid sequences or of two polynucleotide sequences (also referred to herein as nucleic acid sequences), the sequences are aligned for optimal comparison purposes. In order to optimize the alignment between the two sequences gaps may be introduced in any of the two sequences that are compared. Such alignment can be carried out over the full length of the sequences being compared. Alternatively, the alignment may be carried out over a shorter length, for example over about 20, about 50, about 100 or more nucleic acids/based or amino acids. The percent homology or percent identity is the percentage of identical matches between the two sequences over the reported aligned region.

A comparison of sequences and determination of percent identity between two sequences can be accomplished using a mathematical algorithm. The skilled person will be aware of the fact that several different computer programs are available to align two sequences and determine the homology between two sequences (Kruskal, J. B. (1983) An overview of sequence comparison In D. Sankoff and J. B. Kruskal, (ed.), Time warps, string edits and macromolecules: the theory and practice of sequence comparison, pp. 1-44 Addison Wesley). The percent identity between two amino acid sequences or between two nucleotide sequences may be determined using the Needleman and Wunsch algorithm for the alignment of two sequences. (Needleman, S. B. and Wunsch, C. D. (1970) J. Mol. Biol. 48, 443-453). Both amino acid sequences and polynucleotide sequences can be aligned by the algorithm. The Needleman-Wunsch algorithm has been implemented in the computer program NEEDLE. For the purpose of this invention the NEEDLE program from the EMBOSS package was used (version 2.8.0 or higher, EMBOSS: The European Molecular Biology Open Software Suite (2000) Rice, P. Longden, I. and Bleasby, A. Trends in Genetics 16, (6) pp 276-277, http://emboss.bioinformatics.nl/). For protein sequences EBLOSUM62 is used for the substitution matrix. For nucleotide sequence, EDNAFULL is used. The optional parameters used are a gap-open penalty of 10 and a gap extension penalty of 0.5. The skilled person will appreciate that all these different parameters will yield slightly different results but that the overall percentage identity of two sequences is not significantly altered when using different algorithms.

After alignment by the program NEEDLE as described above the percentage of identity between a query sequence and a sequence of the invention is calculated as follows: Number of corresponding positions in the alignment showing an identical aminoacid or identical nucleotide in both sequences divided by the total length of the alignment after subtraction of the total number of gaps in the alignment. The percent identity defined as herein can be obtained from NEEDLE by using the NOBRIEF option and is labelled in the output of the program as “longest-identity”.

The polynucleotide and protein sequences of the present invention can further be used as a “query sequence” to perform a search against public databases to, for example, identify other family members or related sequences. Such searches can be performed using the NBLAST and XBLAST programs (version 2.0) of Altschul, et al. (1990) J. Mol. Biol. 215:403-10. BLAST nucleotide searches can be performed with the NBLAST program, score=100, wordlength=12 to obtain nucleotide sequences homologous to the polynucleotide of the invention. BLAST protein searches can be performed with the XBLAST program, score=50, wordlength=3 to obtain amino acid sequences homologous to protein molecules of the invention. To obtain gapped alignments for comparison purposes, Gapped BLAST can be utilized as described in Altschul et al., (1997) Nucleic Acids Res. 25(17): 3389-3402. When utilizing BLAST and Gapped BLAST programs, the default parameters of the respective programs (e.g., XBLAST and NBLAST) can be used. See the homepage of the National Center for Biotechnology Information at http://www.ncbi.nlm.nih.gov/.

### Lipolytic Enzyme

A lipolytic enzyme, also referred to herein as lipase, is an enzyme that hydrolyses triacylglycerol and/or galactolipid and or phospholipids.

Lipase activity may be determined spectrophotometrically by using the chromogenic substrate p-nitrophenyl palmitate (pNPP, Sigma N-2752). In this assay the pNPP is dissolved in 2-propanol (40 mg pNPP per 10 ml 2-propanol (Merck 1.09634)) and suspended in 100 mM Acetate buffer pH=5.0 containing 1.0% Triton X-100 (Merck 1.12298) (5 ml substrate in 45 ml buffer). The final substrate concentration is 1.1 mM. The lipase is incubated with this substrate solution at 37° C. for 10 minutes. The reaction is stopped by addition of stop buffer 2% TRIS (Merck 1.08387)+1% Triton X-100 in a 1:1 ratio with respect to the reaction mixture and subsequently the formed p-nitrophenol (pNP) is measured at 405 nm. This assay can also be applied at different pH values in order to determine pH dependence of a lipase. It should be understood that at different pH values different buffers might be required or that different detergents might be necessary to emulsify the substrate. One lipase unit is defined as the amount of enzyme that liberates 1 micromole of p-nitrophenol per minute at the reaction conditions stated. It should be understood that it is not uncommon practice in routine analysis to use standard calibration enzyme solutions with known activity determined in a different assay to correlate activity a given assay with units as would be determined in the calibration assay.

Alternatively, lipase activity may be determined by using 2,3-mercapto-1-propanol-tributyrate (TBDMP) as a substrate. Lipase hydrolyses the thioester bond(s) of TBDMP thereby liberating butanoic acid and 2,3-mercapto-1-propanol-dibutyrate, 2,3-mercapto-1-propanol-monobutyrate or 2,3-mercapto-1-propanol. The liberated thiol groups are titrated in a subsequent reaction with 4,4,-dithiodipyridine (DTDP) forming 4-thiopyridone. The latter is in a tautomeric equilibrium with 4-mercapthopyridine which absorbs at 334 nm. The reaction is carried out in 0.1 M acetate buffer pH 5.0 containing 0.2% Triton-X100, 0.65 mM TBDMP and 0.2 mM DTDP at 37° C. One lipase unit is defined as the amount of enzyme that liberates 1 micromole of 4-thiopyridone per minute at the reaction conditions stated.

In addition to spectrophotometric measurement lipase activity may also be determined using titrimetric measurement. For example the esterase activity of a lipolytic enzyme may be measured on tributyrin as a substrate according to Food Chemical Codex, Forth Edition, National Academy Press, 1996, p 803.

A phospholipase is an enzyme that catalyzes the release of fatty acyl groups from a phospholipid. It may be a phospholipase A2 (PLA2, EC 3.1.1.4) or a phospholipase A1 (EC 3.1.1.32). It may or may not have other activities such as triacylglycerol lipase (EC 3.1.1.3) and/or galactolipase (EC 3.1.1.26) activity.

The phospholipase may be a native enzyme from mammalian or microbial sources.

An example of a mammalian phospholipase is pancreatic PLA2, e.g. bovine or porcine PLA2 such as the commercial product Lecitase 10L (porcine PLA2, product of Novozymes A/S).

Microbial phospholipases may be from Fusarium, e.g. F. oxysporum phospholipase A1 (WO 1998/026057), F. venenatum phospholipase A1 (described in WO 2004/097012 as a phospholipase A2 called FvPLA2), from Tuber, e.g. T. borchii phospholipase A2 (called TbPLA2, WO 2004/097012).

The phospholipase may also be a lipolytic enzyme variant with phospholipase activity, e.g. as described in WO 2000/032758 or WO 2003/060112.

The phospholipase may also catalyze the release of fatty acyl groups from other lipids present in the dough, particularly wheat lipids. Thus, the phospholipase may have triacylglycerol lipase activity (EC 3.1.1.3) and/or galactolipase activity (EC 3.1.1.26). The phospholipase may be a lipolytic enzyme as described in WO2009/106575, such as the commercial product Panamore®, product of DSM.

The triacyl glycerol lipase may be a fungal lipase, preferably from Rhizopus, Aspergillus, Candida, Penicillum, Thermomyces, or Rhizomucor. In an embodiment the triacyl glycerol lipase is from Rhyzopus, in a further embodiment a triacyl glycerol lipase from Rhyzopus oryzae is used. Optionally a combination of two or more triacyl glycerol lipases may be used

### Cellulase

A cellulase may be from A. niger or from Trichoderma reesei.

### Amyloglucosidase

The amyloglucosidase, may be an amyloglucosidase from Aspergillus such as from A. oryzae or A. niger, preferably from A. niger.

### Additional Enzyme

The additional enzyme may include without limitation an enzyme as disclosed in WO2006/032281, WO2008/148845, WO2006/012902, WO2006/012899, WO2004/081171 or WO99/43793 or WO2005/066338

### Pre-Mix

The term “pre-mix” is defined herein to be understood in its conventional meaning, i.e. as a mix of baking agents, generally including flour, which may be used not only in industrial bread-baking plants/facilities, but also in retail bakeries. The pre-mix may be prepared by mixing the alpha-amylase polypeptide and the G4-forming amylase or the enzyme composition according to the invention with a suitable carrier such as flour, starch or a salt. The pre-mix may contain additives as mentioned herein.

### Baked Product

The term ‘baked product’ refers to a baked food product prepared from a dough.

Examples of baked products, whether of a white, brown or whole-meal type, which may be advantageously produced by the present invention include bread (in particular white, whole-meal or rye bread), typically in the form of loaves or rolls, French baguette-type bread, pastries, croissants, brioche, panettone, pasta, noodles (boiled or (stir-)fried), pita bread and other flat breads, tortillas, tacos, cakes, pancakes, cookies in particular biscuits, doughnuts, including yeasted doughnuts, bagels, pie crusts, steamed bread, crisp bread, brownies, sheet cakes, snack foods (e.g., pretzels, tortilla chips, fabricated snacks, fabricated potato crisps). The term baked product includes, bread containing from 2 to 30 wt % sugar, fruit containing bread, breakfast cereals, cereal bars, eggless cake, soft rolls and gluten-free bread. Gluten free bread herein and herein after is bread than contains at most 20 ppm gluten. Several grains and starch sources are considered acceptable for a gluten-free diet. Frequently used sources are potatoes, rice and tapioca (derived from cassava) Baked product includes without limitation tin bread, loaves of bread, twists, buns, such as hamburger buns or steamed buns, chapati, rusk, dried steam bun slice, bread crumb, matzos, focaccia, melba toast, zwieback, croutons, soft pretzels, soft and hard bread, bread sticks, yeast leavened and chemically-leavened bread, laminated dough products such as Danish pastry, croissants or puff pastry products, muffins, danish, bagels, confectionery coatings, crackers, wafers, pizza crusts, tortillas, pasta products, crepes, waffles, parbaked products and refrigerated and frozen dough products.

An example of a parbaked product includes, without limitation, partially baked bread that is completed at point of sale or consumption with a short second baking process.

The bread may be white or brown pan bread; such bread may for example be manufactured using a so called American style Sponge and Dough method or an American style Direct method.

The term tortilla herein includes corn tortilla and wheat tortilla. A corn tortilla is a type of thin, flat bread, usually unleavened made from finely ground maize (usually called “corn” in the United States). A flour tortilla is a type of thin, flat bread, usually unleavened, made from finely ground wheat flour. The term tortilla further includes a similar bread from South America called arepa, though arepas are typically much thicker than tortillas. The term tortilla further includes a laobing, a pizza-shaped thick “pancake” from China and an Indian Roti, which is made essentially from wheat flour. A tortilla usually has a round or oval shape and may vary in diameter from about 6 to over 30 cm.

### Dough

The term “dough” is defined herein as a mixture of flour and other ingredients. In one aspect the dough is firm enough to knead or roll. The dough may be fresh, frozen, prepared or parbaked. The preparation of frozen dough is described by Kulp and Lorenz in Frozen and Refrigerated Doughs and Batters.

Dough is made using dough ingredients, which include without limitation (cereal) flour, a lecithin source including egg, water, salt, sugar, flavours, a fat source including butter, margarine, oil and shortening, baker's yeast, chemical leavening systems such as a combination of an acid (generating compound) and bicarbonate, a protein source including milk, soy flour, oxidants (including ascorbic acid, bromate and Azodicarbonamide (ADA)), reducing agents (including L-cysteine), emulsifiers (including mono/di glycerides, monoglycerides such as glycerol monostearate (GMS), sodium stearoyl lactylate (SSL), calcium stearoyl lactylate (CSL), polyglycerol esters of fatty acids (PGE) and diacetyl tartaric acid esters of mono- and diglycerides (DATEM), gums (including guargum and xanthangum), flavours, acids (including citric acid, propionic acid), starch, modified starch, gluten, hum ectants (including glycerol) and preservatives.

Cereals include maize, rice, wheat, barley, sorghum, millet, oats, rye, triticale, buckwheat, quinoa, spelt, einkorn, emmer, durum and kamut.

Dough is usually made from basic dough ingredients including (cereal) flour, such as wheat flour or rice flour, water and optionally salt. For leavened products, primarily baker's yeast is used next to chemical leavening systems such as a combination of an acid (generating compound) and bicarbonate.

The term dough herein includes a batter. A batter is a semi-liquid mixture, being thin enough to drop or pour from a spoon, of one or more flours combined with liquids such as water, milk or eggs used to prepare various foods, including cake.

The dough may be made using a mix including a cake mix, a biscuit mix, a brownie mix, a bread mix, a pancake mix and a crepe mix.

The term dough includes frozen dough, which may also be referred to as refrigerated dough. There are different types of frozen dough; that which is frozen before proofing and that which is frozen after a partial or complete proofing stage. The frozen dough is typically used for manufacturing baked products including without limitation biscuits, breads, bread sticks and croissants.

Synergistic Effect

The combined use of an alpha-amylase polypeptide and a G4-forming amylase has a synergistic effect on reduction of hardness after storage of a baked product and/or reduced loss of resilience over storage of a baked product.

The combination of an alpha-amylase polypeptide and a G4-forming amylase may have a synergistic effect on an improved property as described herein. Such improved property may include, but is not limited to, increased strength of the dough, increased elasticity of the dough, increased stability of the dough, reduced stickiness of the dough, improved extensibility of the dough, improved machineability of the dough, increased volume of the baked product, improved flavour of the baked product, improved crumb structure of the baked product, improved crumb softness of the baked product, reduced blistering of the baked product, improved crispiness, improved resilience both initial and in particular after storage, reduced hardness after storage and/or improved anti-staling of the baked product.

The improved property may include faster dough development time of the dough and/or reduced dough stickiness of the dough.

The improved property may include improved foldability of the baked product, such as improved foldability of a tortilla, a pancake, a flat bread, a pizza crust, a roti and/or a slice of bread.

The improved property may include improved flexibility of the baked product including improved flexibility of a tortilla, a pancake, a flat bread, a pizza crust, a roti and/or a slice of bread.

The improved property may include improved stackability of flat baked products including tortillas, pancakes, flat breads, pizza crusts, roti.

The improved property may include reduced stickiness of noodles and/or increased flexibility of noodles.

The improved property may include reduced clumping of cooked noodles and/or improved flavor of noodles even after a period of storage.

The improved property may include reduction of formation of hairline cracks in a product in crackers as well as creating a leavening effect and improved flavor development.

The improved property may include improved mouth feel and/or improved softness on squeeze,

The improved property may include reduced damage during transport, including reduced breaking during transport.

The improved property may include reduced hardness after storage of gluten-free bread.

The improved property may include improved resilience of gluten-free bread. The improved property may include improved resilience both initial and in particular after storage of gluten-free bread.

The improved property may include reduced hardness after storage of rye bread.

The improved property may include reduced loss of resilience over storage of rye bread.

The improved property may include improved slice ability. This may be demonstrated by observing the amount of crumbs after slicing. Less crumbs indicate a better slice ability

The improved property may include improved crumb structure and/or resilience, without creating gumminess.

The improved property may include reduced loss of resilience over storage of a baked product comprising at least 5 wt % sugar, in an aspect comprising at least 8 wt % sugar, in an aspect comprising at least 12 wt % sugar, in an aspect comprising at least 15 wt % sugar based on flour. In an aspect comprising at least 18 wt % sugar, in an aspect comprising at least 20 wt % sugar, in an aspect comprising at least 25 wt % sugar, in an aspect comprising at least 30 wt % sugar based on flour. So for example 5% means 50 grams sugar per 1000 gram of flour used in the recipe.

The improved property may include reduced hardness after storage of a baked product comprising at least 5 wt % sugar, in an aspect comprising at least 8 wt % sugar, in an aspect comprising at least 12 wt % sugar, in an aspect comprising at least 15 wt % sugar based on flour. In an aspect comprising at least 18 wt % sugar, in an aspect comprising aspect at least 20 wt % sugar, in an aspect comprising at least 25 wt % sugar, in an aspect comprising at least 30 wt % sugar based on flour. So for example 5% means 50 grams sugar per 1000 grams of flour used in the recipe.

A synergistic effect, which may also be referred to as synergy, may be determined by making doughs or baked products with addition of the alpha-amylase polypeptide and the G4-forming amylase separately and in combination, and comparing the effects; synergy is indicated when the combination produces a better effect than each enzyme used separately. The comparison may be made between the combination and each enzyme alone at double dosage (on the basis of the ppm of enzyme added with defined enzyme activity per enzyme or on the basis of enzyme activity added on weight of flour or on weight of endproduct. This synergy may be said to occur if the effect of Y ppm of enzyme A+Z ppm of enzyme B, is greater than the effect with 2Y ppm of enzyme A and also greater than the effect with 2Z ppm of enzyme B.

Thus for example, this synergy may be said to occur if the effect of 50 ppm of enzyme A+5 ppm of enzyme B, is greater than the effect with 100 ppm of enzyme A and also greater than the effect with 10 ppm enzyme B.

Alternatively, the comparison may be made with equal total enzyme dosages (as pure enzyme protein per kg flour or per weight of endproduct). If the effect with the combination is greater than with either enzyme alone, this may be taken as an indication of synergy. As an example, synergy may be said to occur if the effect of 0.5 mg of enzyme A+1.0 mg of enzyme B is greater than the effect with 1.0 mg of enzyme A and also greater than the effect with 2.0 mg of enzyme B.

Suitable dosages for the enzymes may typically be found in the range 0.01-20 mg of enzyme protein per kg of flour particularly 0.1-10 mg/kg. Suitable dosages for each of the two enzymes in the combination may be found by first determining a suitable dosage for each enzyme alone (e.g. the optimum dosage, i.e. the dosage producing the greatest effect) and using 30-67% (e.g. 33-50%, particularly 50%) of that dosage for each enzyme in the combination. Again, if the effect with the combination is greater than with either enzyme used separately, this may be taken as an indication of synergy.

In an embodiment of the enzyme composition according to the invention, said composition comprises an additional enzyme.

In an embodiment of the enzyme composition according to the invention, said composition comprises at least one additional enzyme, in an aspect two additional enzymes, in an aspect three additional enzymes.

The alpha-amylase may be for example combined with the additional enzyme prior to combining it with the G4-forming amylase. Combining may include without limitation mixing or adding jointly to dough ingredients.

The additional enzyme may be an enzyme as described in U.S. Pat. No. 4,598,048 which describes the preparation of a maltogenic amylase enzyme.

In an embodiment of the enzyme composition according to the invention, the additional enzyme is selected from the group consisting of an amylase, a further alpha-amylase, beta-amylase, a cyclodextrin glucanotransferase, a protease, a peptidase, a transglutaminase, a triacyl glycerol lipase, a galactolipase, a phospholipase, a cellulase, a hem icellulase, a protease, a protein disulfide isomerase, a glycosyltransferase, a peroxidase, a laccase, an oxidase, a hexose oxidase, a glucose oxidase, a aldose oxidase, a pyranose oxidase, a lipoxygenase, a L-amino acid oxidase and an amyloglucosidase.

In an embodiment of the enzyme composition according to the invention the additional enzyme is a lipolytic enzyme, preferably a phospholipase, a galactolipase or an enzyme having both phospholipase and galactolipase activity.

In an embodiment of the enzyme composition according to the invention the additional enzyme is a phospholipase.

In an embodiment of the enzyme composition according to the invention the additional enzyme is a galactolipase.

In an embodiment of the enzyme composition according to the invention the additional enzyme is an enzyme having both phospholipase and galactolipase activity

In an embodiment of the enzyme composition according to the invention the additional enzyme is Panamore® as described in WO2009/106575.

In an embodiment of the enzyme composition of the invention the additional enzyme is an enzyme as described in WO9826057.

In an aspect of the enzyme composition according to the invention the additional enzyme is an enzyme as described in U.S. RE38,507.

In an aspect of the enzyme composition according to the invention the additional enzyme is an enzyme as described in WO 9943794, in particular in as defined in claim 1 of EP1058724B1.

In an aspect of the enzyme composition according to the invention the additional enzyme is an enzyme as described in WO2008/148845

In an aspect of the enzyme composition according to the invention the additional enzyme is an enzyme as described in WO2006/032281

Suitable additional enzymes may be amylases as described in WO2008/148845 which are a polypeptides according to SEQ ID NO:1 as defined in WO2008/148845 or a variant of SEQ ID NO:1 as defined in WO2008/148845 comprising one or more amino acid substitutions including but not limited to any one of the following positions: 261 and 288 said positions being defined with reference to SEQ ID NO:1 as defined in W02008/148845.

A suitable additional enzyme may be a fungal amylase, including Bakezyme® P 500 BG (DSM, The Netherlands).

A suitable additional enzyme may be a hemicellulase, including Bakezyme® HSP (DSM, The Netherlands) 6000 BG (DSM, The Netherlands) and/or Bakezyme® BXP5001 (DSM, The Netherlands).

If one or more additional enzyme activities are to be added in accordance with the methods of the present invention, these activities may be added separately or together with the enzyme composition according to the invention or pre-mix according to the invention. The other enzyme activities may be dosed in accordance with established baking practices.

Preferably the enzyme composition according to the invention is provided in a dry form, to allow easy handling of the product. Irrespective of the formulation of the enzyme, the enzyme composition according to the invention may comprise one or more components selected from the group consisting of milk powder, gluten, granulated fat, an additional enzyme, an amino acid, a salt, an oxidant such as ascorbic acid, bromate and azodicarbonamide, a reducing agent such as L-cysteine, an emulsifier such as mono-glycerides such as glycerol monostearate, di-glycerides (or combinations thereof), sodium stearoyl lactylate, calcium stearoyl lactylate, polyglycerol esters of fatty acids and diacetyl tartaric acid esters of mono- and diglycerides, gums such as guargum and xanthangum, flavours, acids such as citric acid and propionic acid, starch, modified starch, gluten, humectants such as glycerol, and preservatives.

For inclusion in a pre-mix of flour it is advantageous that the enzyme composition according to the invention is in the form of a dry product, e.g., a non-dusting granulate, whereas for inclusion together with a liquid it is advantageously in a liquid form.

The invention further concerns a pre-mix comprising flour, an alpha-amylase polypeptide and a G4-forming amylase.

In an embodiment of the pre-mix according to the invention, the pre-mix further comprises one or more components selected from the group consisting of milk powder, gluten, granulated fat, an additional enzyme, an amino acid, a salt, an oxidant such as ascorbic acid, bromate and azodicarbonamide, a reducing agent such as L-cysteine, an emulsifier such as mono-glycerides (such as glycerol monostearate), di-glycerides (or combinations thereof), sodium stearoyl lactylate, calcium stearoyl lactylate, polyglycerol esters of fatty acids and diacetyl tartaric acid esters of mono- and diglycerides, gums such as guargum and xanthangum, flavours, acids such as citric acid and propionic acid, starch, modified starch, gluten, humectants such as glycerol, and preservatives.

In an embodiment of the pre-mix according to the invention the additional enzyme is selected from the group consisting of an amylase, a further alpha-amylase, beta-amylase, a cyclodextrin glucanotransferase, a protease, a peptidase, a transglutaminase, a triacyl glycerol lipase, a galactolipase, a phospholipase, a cellulase, a hem icellulase, a protease, a protein disulfide isomerase, a glycosyltransferase, a peroxidase, a laccase, an oxidase, a hexose oxidase, a glucose oxidase, a aldose oxidase, a pyranose oxidase, a lipoxygenase, a L-amino acid oxidase and an amyloglucosidase.

In an embodiment of the pre-mix according to the invention the additional enzyme is a lipolytic enzyme, preferably a phospholipase, a galactolipase or an enzyme having both phospholipase and galactolipase activity.

In an embodiment of the pre-mix according to the invention the additional enzyme is a phospholipase.

In an embodiment of the pre-mix according to the invention the additional enzyme is a galactolipase.

In an embodiment of the pre-mix according to the invention the additional enzyme is an enzyme having both phospholipase and galactolipase activity.

The invention further relates to a method to prepare a dough comprising combining the alpha-amylase polypeptide and the G4-forming amylase and at least one dough ingredient.

A dough ingredient includes a component selected from flour, egg, water, salt, sugar, flavours, fat (including butter, margarine, oil and shortening), baker's yeast, a chemical leavening system, milk, oxidants (including ascorbic acid, bromate and Azodicarbonamide (ADA)), reducing agents (including L-cysteine), emulsifiers (including mono/di glycerides, mono glycerides such as glycerol monostearate (GMS), sodium stearoyl lactylate (SSL), calcium stearoyl lactylate (CSL), polyglycerol esters of fatty acids (PGE) and diacetyl tartaric acid esters of mono- and diglycerides (DATEM), gums (including guargum and xanthangum), acids (including citric acid, propionic acid), starch, modified starch, gluten, humectants (including glycerol) and preservatives.

‘Combining’ includes without limitation, adding the alpha-amylase and the G4-forming amylase to the at least one dough ingredient, adding the at least one dough ingredient adding the alpha-amylase and the G4-forming amylase, and includes mixing the alpha amylase, the G4-forming amylase and the at least one dough ingredient.

One or more additional enzymes may also be incorporated into the dough In an embodiment, the additional enzyme may be an amylase, including a further alpha-amylase, such as a fungal alpha-amylase (which may be useful for providing sugars fermentable by yeast and retarding staling), beta-amylase, a cyclodextrin glucanotransferase, a protease, a peptidase, in particular, an exopeptidase (which may be useful in flavour enhancement), transglutaminase, triacyl glycerol lipase (which may be useful for the modification of lipids present in the dough or dough constituents so as to soften the dough), galactolipase, phospholipase, cellulase, hemicellulase, in particular a pentosanase such as xylanase (which may be useful for the partial hydrolysis of pentosans, more specifically arabinoxylan, which increases the extensibility of the dough), protease (which may be useful for gluten weakening in particular when using hard wheat flour), protein disulfide isomerase, e.g., a protein disulfide isomerase as disclosed in WO 95/00636, glycosyltransferase, peroxidase (which may be useful for improving the dough consistency), laccase, or oxidase, hexose oxidase, e.g., a glucose oxidase, aldose oxidase, pyranose oxidase, lipoxygenase or L-amino acid oxidase (which may be useful in improving dough consistency) or a protease.

The method to prepare a dough according to the invention comprises combining the alpha-amylase polypeptide and the G4-forming amylase and at least one dough ingredient.

‘Combining’ includes without limitation, adding the alpha-amylase polypeptide and the G4-forming amylase to the at least one dough ingredient, adding the at least one dough ingredient to the alpha-amylase polypeptide and the G4-forming amylase, and includes mixing of the alpha-amylase, the G4-forming amylase and the at least one dough ingredient

In an embodiment of the method according to the invention to prepare a dough, the method comprises the step of combining the enzyme composition according to the invention or the pre-mix according to the invention and at least one dough ingredient.

‘Combining’ includes without limitation, adding the enzyme composition according to the invention or the pre-mix according to the invention to at least one dough ingredient, adding at least one dough ingredient to the enzyme composition according to the invention or to the pre-mix according to the invention, and includes mixing of the enzyme composition according to the invention or the pre-mix according to the invention and at least one dough ingredient.

The invention also relates to a dough comprising the alpha-amylase polypeptide and the G4-forming amylase, the enzyme composition as defined in any one of claims 1 to 3 or the pre-mix as defined in any one of claims 4 to 6.

The preparation of a dough from the dough ingredients is well known in the art and includes mixing of said ingredients and optionally one or more moulding and fermentation steps.

The method according to the invention to prepare a baked product comprises the step of baking the dough according to the invention.

The preparation of baked products from such doughs is also well known in the art and may comprise moulding and shaping and further fermentation of the dough followed by baking at required temperatures and baking times. In one embodiment the invention provides a method to prepare a baked product comprising the step of baking the dough according to the invention. The baking of the dough to produce a baked product may be performed using methods well known in the art. The invention also provides a baked product obtainable according to this method. In an embodiment the baked product according to the invention is bread or cake. In one aspect of the invention, the enzyme composition according to the invention may be used to prepare laminated doughs for baked products with improved crispiness.

In an embodiment of the method to prepare a baked product, the method comprises baking a dough comprising the enzyme composition according to the invention or pre-mix according to the invention.

In an embodiment of the method to prepare a baked product, the method comprises baking a dough comprising the pre-mix according to the invention.

In an embodiment of the method to prepare a baked product the baked product is bread or cake.

The present invention also relates to methods for preparing a dough or a baked product comprising incorporating into the dough an effective amount of the alpha-amylase polypeptide and the G4-forming amylase, which improves one or more properties of the dough or the baked product obtained from the dough relative to a dough or a baked product in which the alpha-amylase polypeptide and the G4-forming amylase are not incorporated.

The phrase “incorporating into the dough” is defined herein as adding the alpha-amylase polypeptide and the G4-forming amylase to the dough, any ingredient from which the dough is to be made, and/or any mixture of dough ingredients from which the dough is to be made. In other words, the alpha-amylase polypeptide and the G4-forming amylase may be added in any step of the dough preparation and may be added in one, two or more steps. The alpha-amylase polypeptide and the G4-forming amylase are added to the ingredients of a dough that is kneaded and baked to make the baked product using methods well known in the art. See, for example, U.S. Pat. No. 4,567,046, EP-A-426,211, JP-A-60-78529, JP-A-62-111629, and JP-A-63-258528.

The term “effective amount” is defined herein as an amount of the alpha-amylase polypeptide or G4-forming amylase that is sufficient for providing a measurable effect on at least one property of interest of the dough and/or baked product. A suitable amount of alpha-amylase is in a range of 0.5-1500 NBAU/kg flour, in an embodiment 5-200 NBAU/kg flour, in a further embodiment 20-100 NBAU/kg flour. A suitable amount includes 1 ppm-2000 ppm of an enzyme having an activity in a range of about 700 to 1100 NBAU/g. In an embodiment an effective amount is in a range of 10-200 ppm of an enzyme having an activity in a range of about 700 to 1100 NBAU/g, in another embodiment 30-100 ppm of an enzyme having an activity in a range of about 700 to 1100 NBAU/g. In an embodiment an effective amount is in a range of 10-200 ppm of an enzyme having an activity of about 700 to 1100 NBAU/g. Herein and hereinafter NBAU stands for New Baking Amylase Unit as defined in the examples under the heading NBAU Assay as described herein.

The term “improved property” is defined herein as any property of a dough and/or a product obtained from the dough, particularly a baked product, which is improved by the action of the alpha-amylase polypeptide in combination with the G4-forming amylase, the enzyme composition according to the invention or the pre-mix according to the invention relative to a dough or product in which the alpha-amylase polypeptide in combination with the G4-forming amylase are not incorporated. The improved property may include, but is not limited to, increased strength of the dough, increased elasticity of the dough, increased stability of the dough, reduced stickiness of the dough, improved extensibility of the dough, improved machineability of the dough, increased volume of the baked product, improved flavour of the baked product, improved crumb structure of the baked product, improved crumb softness of the baked product, reduced blistering of the baked product, improved crispiness, improved resilience both initial and in particular after storage, reduced hardness after storage and/or improved anti-staling of the baked product.

The improved property may include faster dough development time of the dough and/or reduced dough stickiness of the dough.

The improved property may include improved foldability of the baked product, such as improved foldability of a tortilla, a pancake, a flat bread, a pizza crust, a roti and/or a slice of bread.

The improved property may include improved flexibility of the baked product including improved flexibility of a tortilla, a pancake, a flat bread, a pizza crust, a roti and/or a slice of bread.

The improved property may include improved stackability of flat baked products including tortillas, pancakes, flat breads, pizza crusts, roti.

The improved property may include reduced stickiness of noodles and/or increased flexibility of noodles.

The improved property may include reduced clumping of cooked noodles and/or improved flavor of noodles even after a period of storage.

The improved property may include reduction of formation of hairline cracks in a product in crackers as well as creating a leavening effect and improved flavor development.

The improved property may include improved mouth feel and/or improved softness on squeeze,

The improved property may include reduced damage during transport, including reduced breaking during transport.

The improved property may include reduced hardness after storage of gluten-free bread.

The improved property may include improved resilience of gluten-free bread. The improved property may include improved resilience both initial and in particular after storage of gluten-free bread.

The improved property may include reduced hardness after storage of rye bread.

The improved property may include reduced loss of resilience over storage of rye bread,

The improved property may include reduced loss of resilience over storage of a baked product comprising at least 5 wt % sugar, in an aspect comprising at least 8 wt % sugar, in an aspect comprising at least 12 wt % sugar, in an aspect comprising at least 15 wt % sugar based on flour. In an aspect comprising at least 18 wt % sugar, in an aspect comprising at least 20 wt % sugar, in an aspect comprising at least 25 wt % sugar, in an aspect comprising at least 30 wt % sugar based on flour. So for example 5% means 50 grams sugar per 1000 gram of flour used in the recipe.

The improved property may include reduced hardness after storage of a baked product comprising at least 5 wt % sugar, in an aspect comprising at least 8 wt % sugar, in an aspect comprising at least 12 wt % sugar, in an aspect comprising at least 15 wt % sugar based on flour. In an aspect comprising at least 18 wt % sugar, in an aspect comprising aspect at least 20 wt % sugar, in an aspect comprising at least 25 wt % sugar, in an aspect comprising at least 30 wt % sugar based on flour. So for example 5% means 50 grams sugar per 1000 gram of flour used in the recipe.

Improved mouth feel includes sense of softness on an initial bite or after chewing, preferably without a sticky feeling in the mouth and/or without the baked product sticking to the teeth. Improved mouth feel includes the baked product feeling less dry in the mouth on an initial bite or after chewing. Improved mouth feel includes the baked product feeling less dry in the mouth on an initial bite or after chewing after it has been kept outside its packaging or container. The improved property may include that after a slice of bread was taken from its packaging or container and exposed to ambient conditions for 5 minutes, in an aspect for 10 minutes, in an aspect for 20 minutes it has improved mouthfeel.

The improved property may include that after a the cookie was taken from its packaging or container and exposed to ambient conditions for 10 minutes, in an aspect for 20 minutes, in an aspect for 30 minutes, in an aspect an hour it has improved mouthfeel.

In an aspect ambient conditions herein and herein after include a temperature of 20 degrees C. and a moisture level of 40% humidity.

Reduced breaking during transport includes the baked product, including without limitation cookies, bread such as gluten free bread, does not break in additional pieces as a consequence of transport.

Improved softness on squeeze includes the tactile experience that if a bun is held between the fingers and the thumb of a hand and the thumb and fingers are moved towards each other it takes less force.

Improved foldability of a baked product may be determined as follows.

The baked product is laid on a flat surface. The baked product is folded by picking up one edge of the product and placing it on the opposite edge of the product.

This way a folded baked product is obtained having a bend curve in an area located at or close to the center. The surface of the outside of the bend of folded baked product is visually inspected. The foldability is improved if fewer cracks are observed at or close to the bend. Foldability is improved if the folded baked product has less tendency to break along the bend compared with a control bread. Foldability is improved if the folded baked product is less damaged along the bend compared with a control bread. This may be a particularly useful property if the baked product is a tortilla, a pancake and/or a slice of bread.

Improved stackability may be determined as follows.

10 baked products are stacked on top of each other and sealed in a polymer package, such as polyethylene foil. This yields a pack of baked products. 10 packs of baked product are stacked on top of each other and kept under ambient conditions for 3 days, in an aspect for 5 days in an aspect for 1 week, in an aspect for 2 weeks. Ambient conditions are conditions as defined herein. After this period the bottom pack of baked products is opened, the baked products are separated from each other and the surfaces of the products are visually inspected. The stackability is improved if less surface damage is observed. Surface damage may be caused e.g. by rupture of the surface during separation of two baked products that were stacked on top of each other. This may be a particularly useful property if the baked product is a tortilla.

Faster dough development time may be determined as follows

Dough development time is the time the dough need to reach maximum consistency, maximum viscosity before gluten strands begin to break down. It may be determined by measuring peak time, using a Farinograph® from Brabender®, Germany. If a faronigraph is used to determine dough development time, dough development time is the time between the moment water is added and the moment the curve reaches its highest point. Peak time is preferably expressed in minutes.

Reduced dough stickiness may be determined as follows.

Dough stickiness is preferably determined on two separate batches of at least 8 dough pieces, with the Texture Analyser TAXT2i (Stable Micro Systems Ltd., Surrey, UK) equipped with a 5 kg load cell in the measure force in compression mode with a cylindrical probe (25 mm diameter). Using pre- and post-test speeds of 2.0 mm/s, while the test speed is 1.0 mm/s. Dough pieces are centered and compressed 50% and the probe is held for 10 s at maximum compression. A negative peak value indicates dough stickiness. A less negative peak value indicates reduced dough stickiness.

Increased flexibility may be determined as follows.

The baked product is laid on a flat surface. The baked product is rolled to a shape similar to a pipe, this way a rolled baked product is obtained. The flexibility is improved if the rolled baked product remains its rolled up shape and does not roll open. This may be a particularly useful property if the baked product is a tortilla or a pancake.

The improved property may be determined by comparison of a dough and/or a baked product prepared with and without addition of the (isolated) polypeptide of the present invention in accordance with the methods of present invention which are described below in the Examples. Organoleptic qualities may be evaluated using procedures well established in the baking industry, and may include, for example, the use of a panel of trained taste-testers.

The term “increased strength of the dough” is defined herein as the property of a dough that has generally more elastic properties and/or requires more work input to mould and shape.

The term “increased elasticity of the dough” is defined herein as the property of a dough which has a higher tendency to regain its original shape after being subjected to a certain physical strain.

The term “increased stability of the dough” is defined herein as the property of a dough that is less susceptible to forming faults as a consequence of mechanical abuse thus better maintaining its shape and volume and is evaluated by the ratio of height: width of a cross section of a loaf after normal and/or extended proof.

The term “reduced stickiness of the dough” is defined herein as the property of a dough that has less tendency to adhere to surfaces, e.g., in the dough production machinery, and is either evaluated empirically by the skilled test baker or measured by the use of a texture analyser (e.g. a TAXT Plus) as known in the art.

The term “improved extensibility of the dough” is defined herein as the property of a dough that can be subjected to increased strain or stretching without rupture.

The term “improved machineability of the dough” is defined herein as the property of a dough that is generally less sticky and/or more firm and/or more elastic. Consequently there is less fouling of plant equipment and a reduced need for cleaning.

The term “increased volume of the baked product” is preferably measured as the volume of a given loaf of bread determined by an automated bread volume analyser (eg. BVM-3, TexVol Instruments AB, Viken, Sweden), using ultrasound or laser detection as known in the art. In case the volume is increased, the property is improved. Alternatively the height of the baked product after baking in the same size tin is an indication of the baked product volume. In case the height of the baked product has increased, the volume of the baked product has increased.

The term “reduced blistering of the baked product” is defined herein as a visually determined reduction of blistering on the crust of the baked bread.

The term “improved crumb structure of the baked product” is defined herein as the property of a baked product with finer cells and/or thinner cell walls in the crumb and/or more uniform/homogenous distribution of cells in the crumb and is usually evaluated visually by the baker or by digital image analysis as known in the art (eg. C-cell, Calibre Control International Ltd, Appleton, Warrington, UK).

The term “improved softness of the baked product” is the opposite of “hardness” and is defined herein as the property of a baked product that is more easily compressed and is evaluated either empirically by the skilled test baker or measured by the use of a texture analyzer (e.g. TAXT Plus) as known in the art.

The term “improved flavor of the baked product” is evaluated by a trained test panel.

The term “improved anti-staling of the baked product” is defined herein as the properties of a baked product that have a reduced rate of deterioration of quality parameters, e.g. reduced hardness after storage and/or decreased loss of resilience after storage.

Anti-staling properties may be demonstrated by a reduced hardness after storage of the baked product. The enzyme composition according to the invention or the pre-mix according to the invention may result in reduced hardness, e.g. in a baked product that is more easily compressed. The hardness of the baked product may be evaluated either empirically by the skilled test baker or measured by the use of a texture analyzer (e.g. TAXT Plus) as known in the art. The hardness measured within 24 hours after baking is called initial hardness. The hardness measured 24 hours or more after baking is called hardness after storage, and is also a measure for determining shelf life. In case the initial hardness has reduced, it has improved. In case the hardness after storage has reduced, it has improved. Preferably hardness is measured as described in example 9 herein. Resilience of the baked product is preferably measured by the use of a texture analyzer (e.g. TAXTPlus) as known in the art.

The resilience measured within 24 hours after baking is called initial resilience. The resilience measured 24 hours or more after baking is called resilience after storage, and is also a measure for determining shelf life. Freshly baked product typically gives crumb of high initial resilience but resilience is lost over shelf-life. Improved anti-staling properties may be demonstrated by a reduced loss of resilience over storage. Preferably resilience is measured as described in example 1 herein.

The term “improved crispiness” is defined herein as the property of a baked product to give a crispier sensation than a reference product as known in the art, as well as to maintain this crispier perception for a longer time than a reference product. This property can be quantified by measuring a force versus distance curve at a fixed speed in a compression experiment using e.g. a texture analyzer TA-XT Plus (Stable Micro Systems Ltd, Surrey, UK), and obtaining physical parameters from this compression curve, viz. (i) force of the first peak, (ii) distance of the first peak, (iii) the initial slope, (iv) the force of the highest peak, (v) the area under the graph and (vi) the amount of fracture events (force drops larger than a certain preset value). Indications of improved crispness are a higher force of the first peak, a shorter distance of the first peak, a higher initial slope, a higher force of the highest peak, higher area under the graph and a larger number of fracture events. A crispier product should score statistically significantly better on at least two of these parameters as compared to a reference product. In the art, “crispiness” is also referred to as crispness, crunchiness or crustiness, meaning a material with a crispy, crunchy or crusty fracture behaviour.

The present invention may provide a dough having at least one of the improved properties selected from the group consisting of increased strength, increased elasticity, increased stability, reduced stickiness, and/or improved extensibility of the dough.

The invention also may provide a baked product having increased loaf volume. The invention may provide as well a baked product having at least one improved property selected from the group consisting of increased volume, improved flavour, improved crumb structure, improved crumb softness, improved crispiness, reduced blistering and/or improved anti-staling.

The enzyme composition according to the invention or the pre-mix according to the invention may be used for retarding staling of a baked product such as bread and/or cake. Retarding of staling may be indicated by a reduced hardness, in particular a reduced hardness after storage compared to a baked product, including bread and cake, that is produced without the alpha-amylase polypeptide and the G4-forming amylase.

The baked product according to the invention is obtainable by the method according to the invention to prepare the baked product.

### Use

The invention also relates to the use of the enzyme composition according to the invention or the pre-mix according to the invention in a number of industrial processes. Despite the long-term experience obtained with these processes, the enzyme composition or pre-mix according to the invention may feature advantages over the compositions or pre-mixes currently used. Depending on the specific application, these advantages may include aspects like lower production costs, higher specificity towards the substrate, less antigenic, less undesirable side activities, higher yields when produced in a suitable microorganism, more suitable pH and temperature ranges, better tastes of the final product as well as food grade and kosher aspects.

The enzyme composition according to the invention or the pre-mix according to the invention may be used in the food industry, including in food manufacturing. An example of an industrial application is in food, is its use in baking applications. For example to improve quality of the dough and/or the baked product.

In an embodiment of the use in food manufacturing, the use is the manufacture of a baked product, including without limitation a bread or a cake.

In an aspect the use according to the inventions relates to use of an alpha-amylase polypeptide in combination with a G4-forming amylase to reduce hardness after storage of a baked product and/or to reduce loss of resilience over storage of a baked product.

In an aspect the use according to the invention relates to use of an enzyme composition according to the invention or the pre-mix according to the invention to reduce hardness after storage of a baked product and/or to reduce loss of resilience over storage of a baked product.

In an aspect the use according to the invention relates to use of an enzyme composition according to the invention or the pre-mix according to the invention to improve foldability a baked product. In an aspect the use according to the invention relates to use of an enzyme composition according to the invention or the pre-mix according to the invention to improve foldability of a tortilla, a pancake, a flat bread, a pizza crust, a roti and/or a slice of bread, in particular of a tortilla, a pancake and/or a slice of bread.

In an aspect the use according to the invention relates to use of an alpha-amylase polypeptide as described herein combination with a G4-forming amylase as described herein to improve foldability a baked product. In an aspect the use according to the invention relates to use of an enzyme composition according to the invention or the pre-mix according to the invention to improve foldability of a tortilla, a pancake, a flat bread, a pizza crust, a roti and/or a slice of bread, in particular of a tortilla, a pancake and/or a slice of bread

Use of an alpha-amylase polypeptide in combination with a G4-forming amylase as, to reduce hardness after storage of a baked product and/or to reduced loss of resilience over storage of a baked product.

Use of an enzyme composition according to claim any one of claim 1, 3 or 4 or the pre-mix according to any one of claims 5 to 7 referring to any one of claim 1, 3 or 4, to reduce hardness after storage of a baked and/or to reduced loss of resilience over storage of a baked product.

In an aspect, the enzyme composition according to the invention or the pre-mix according to the invention may be used in the production of cake and in the production of a batter from which a cake can be made.

The alpha-amylase polypeptide in combination with the G4 forming amylase, the enzyme composition or the premix according to the invention may be used in the preparation of a wide range of cakes, including shortened cakes, such as for example pound cake and butter cake, and including foam cakes, such as for example meringues, sponge cake, biscuit cake, roulade, genoise and chiffon cake. Sponge cake is a type of soft cake based on wheat flour, sugar, baking powder and eggs (and optionally baking powder). The only fat present is from the egg yolk, which is sometimes added separately from the white. It is often used as a base for other types of cakes and desserts. A pound cake is traditionally prepared from one pound each of flour, butter, eggs, and sugar, optionally complemented with baking powder. In chiffon cake the butter/margarine has been replaced by oil. Sugar and egg yolk is decreased compared to pound or sponge cake and egg white content is increased.

A method to prepare a batter preferably comprises the steps of:

a. preparing the batter of the cake by adding at least:
i. sugar;
ii. flour;
iii. the alpha-amylase polypeptide and the G4-forming amylase;
iv. at least one egg; and
v. optionally a phospholipase.

A method to prepare a cake according to the invention further comprises the step of

b. baking the batter to yield a cake.

The person skilled in the art knows how to prepare a batter or a cake starting from dough ingredients. Optionally one or more other ingredients can be present in the composition e.g. to allow reduction of eggs and/or fat in the cake, such as hydrocolloids, yeast extract, emulsifiers, calcium.

The above-mentioned industrial applications of the enzyme composition according to the invention or pre-mix according to the invention comprise only a few examples and this listing is not meant to be restrictive.

Other uses of the enzyme composition according to the invention or pre-mix according to the invention may include:
the production of glucose, fructose and maltose syrups;
production of starch hydrolysates such as maltodextrins;
production of modified starches;
modification of starch components in animal feed;
replacement of malt in brewing;
use in a glue, including wall paper paste;
use in plastic objects made using starch, including plastic bags made from polymerized starch films; and/or
use in waste bread reprocessing.

### EXAMPLES

### Maltotriose Assay

This assay may be used to determine Activity on maltotriose substrate.

One Maltotriose Unit (MU) is defined as the amount of enzyme that liberates 1 μmole glucose per minute using maltotriose substrate under the following assay conditions. Enzymatic activity was determined in a 30 minutes incubation at 37° C. and pH 5.0 using maltotriose as substrate. Enzymatic hydrolysis of maltotriose results in quantitative release of glucose, which is a measure for enzymatic activity.

Samples of approximately 0.4-4 mg/ml protein were diluted to a range between 0.0125 and 0.125 MU/ml in 100 mM citric acid buffer containing 1 g/L BSA, adjusted to pH 5.0 using 4 N NaOH. 10 mg/ml maltotriose substrate was prepared in 2.5 mM NaCl in MQ water. 160 microliter substrate was preheated for approximately 30 minutes in a PCR thermocycler set at 37° C. in a 96 wells PCR plate. 40 microliter of diluted sample was added to the preheated substrate in the thermocycler and mixed well by pipetting up and down several times. 30 minutes after sample addition, 20 microliter of 0.33 N NaOH was added and mixed well to terminate the reaction, and the PCR plate was taken out of the thermocycler. Released glucose was measured by incubation of 55 microliter of the terminated reaction mixture with 195 microliter of hexokinase monoreagent (Ecoline Glucose Hexokinase FS, DiaSys Diagnostic systems GmbH, Holzheim, Germany) for 15 minutes at room temperature in a flat bottom 96 wells plate. Air bubbles were removed from the surface by centrifugation, after which the absorbance at 340 nm was read using a microtiter plate reader. The amount of glucose released was determined relative to a glucose calibration line.

### Assay to Determine G4-Forming Amylase Activity

The following may for example be used to characterize a G4-forming amylase, either the parent or a variant thereof.

By way of initial background information, waxy maize amylopectin (obtainable as WAXILYS 200 from Roquette, France) is a starch with a very high amylopectin content (above 90%). 20 mg/ml of waxy maize starch is boiled for 3 min. in a buffer of 50 mM MES (2-(N-morpholino) ethanesulfonic acid), 2 mM calcium chloride, pH 6.0 and subsequently incubated at 50° C. and used within half an hour.

One unit of G4-forming amylase is defined as the amount of enzyme which releases hydrolysis products equivalent to 1 μmol of reducing sugar per min. when incubated at 50° C. in a test tube with 4 ml of 10 mg/ml waxy maize starch in 50 mM MES, 2 mM calcium chloride, pH 6.0 prepared as described above. Reducing sugars are measured using maltose as standard and using the dinitrosalicylic acid method of Bernfeld, Methods Enzymol., (1954), 1, 149-158 or another method known in the art for quantifying reducing sugars.

The hydrolysis product pattern of the G4-forming amylase is determined by incubating 0.7 units of G4-forming amylase for 15 or 30 min. at 50° C. in a test tube with 4 ml of 10 mg/ml waxy maize starch in the buffer prepared as described above. The reaction is stopped by immersing the test tube for 3 min. in a boiling water bath. The hydrolysis products are analyzed and quantified by anion exchange HPLC using a Dionex PA 100 column with sodium acetate, sodium hydroxide and water as eluents, with pulsed amperometric detection and with known linear maltooligosaccharides of from glucose to maltoheptaose as standards. The response factor used for maltooctaose to maltodecaose is the response factor found for maltoheptaose.

Alternatively expressed, the G4-forming amylase has the ability in a waxy maize starch incubation test to yield hydrolysis product(s) that would consist of one or more linear maltooligosaccharides of from two to ten D-glucopyranosyl units and optionally glucose, said hydrolysis products being capable of being analysed by anion exchange; such that at least 60%, preferably at least 70%, more preferably at least 80% and most preferably at least 85% by weight of the said hydrolysis product(s) would consist of linear maltooligosaccharides of from three to ten D-glucopyranosyl units, preferably of linear maltooligosaccharides consisting of from four to eight D-glucopyranosyl units.

As used herein, the term “linear malto-oligosaccharide” is used in the normal sense as meaning 2-10 units of α-D-glucopyranose linked by an α-(1->4) bond.

The hydrolysis products can be analysed by any suitable means. For example, the hydrolysis products may be analysed by anion exchange HPLC using a Dionex PA 100 1000 column with pulsed amperometric detection and with, for example, known linear maltooligosaccharides of from glucose to maltoheptaose as standards.

### NBAU Assay

Enzymatic activity of mature DSM-AM is expressed as NBAU. One NBAU is defined as the amount of enzyme resulting in the release of 1 μmole of pNP (para-nitrophenol) per minute using the end blocked pNP-G7 Ceralpha substrate at pH=5.2 and T=37° C.

The principle of the NBAU activity test originates from a (manual) Megazyme α-amylase kit test (Ceralpha). The assay was made suitable for analyzer application. The assay is executed at pH 5.20 taking into account the pH optima for α-glucosidase and amyloglucosidase (pH range 5-6). The test is performed with a Konelab Arena 30 analyzer (Thermo Scientific, Vantaa, Finland).

The enzymatic activity is determined at 37° C. and pH 5.20 using a non-reducing-end blocked p-nitrophenyl maltoheptaoside substrate (=BPNPG7, Ceralpha) combined with excess levels of thermostable α-glucosidase and amyloglucosidase (both from Ceralpha: α-Amylase Reagent R-CAAR4, Megazyme, Ireland). Hydrolysis of the BPNPG7 substrate by an alpha-amylase results in p-nitrophenyl maltosaccharide fragments. The reaction is terminated (and colour developed) by the addition of an alkaline solution. The absorbance at a wavelength of 405 nm is determined and is a measure for enzymatic activity. Activity is calculated from a molar extinction coefficient determination, through a calibration with a para-nitrophenol solution of known concentration.

### Example 1

### Baking Experiment

The baking performance of the mature DSM-AM, PowerFresh Bread 8100 (DuPont Industrial Biosciences, Denmark) and a combination of these enzymes was tested in American style Sponge and Dough white bread. The ingredients are listed in Table 1. The results are listed in Tables 2 and 3 The Control in these tables refers to a loaf of bread prepared according to the same recipe while not containing mature DSM-AM, Powerfresh Bread 8100 (an example of a G-4 forming amylase from Dupont, USA) or a combination thereof.

Mature DSM-AM, may be produced as described in not yet published U.S. patent application Ser. No. 13/532,072. Mature DSM-AM may be produced as described in U.S. Pat. No. 8,426,182 B1.

The ingredients of the sponge listed in Table 1 were mixed in a Hobart A-120 mixer with hook agitator for two minutes at speed one, thereafter for three minutes at speed two, to a final sponge temperature of 24° C. Afterwards the sponge was allowed to ferment for 3 hours at 38° C. in a proof box. After this the ingredients of the dough listed in Table 1 were mixed in the same Hobart A-120 mixer with hook agitator for 30 seconds at speed one. After this the sponge was added to the dough and mixed for another two minutes at speed one, followed by another 8 minutes at speed two to optimum gluten development, to a final dough temperature of 27° C. The fully mixed dough was allowed to rest, covered under plastic, for two minutes at room temperature

The dough was divided in pieces of 565 g, rounded and allowed to rest for 5 minutes at room temperature. Afterwards the dough pieces were moulded using a Unic moulder (top 6.5/bottom 6) and the moulded loaves were placed into bread pans and placed in a proofing cabinet at 38° C. at relative humidity of 85% for 85 minutes. The fully proofed dough pieces were placed in a BeCOM oven and baked in 20 minutes at 215° C. Thereafter the breads were taken out of the oven, depanned and placed on a rack to cool for at least 1 hour at ambient temperature, which is typically between 20 and 25° C. After 1-2 hours cooling, the breads were wrapped in polyethylene plastic bags.

Thereafter the breads were assessed.

The Consistency, Body, Development, Extensibility, Elasticity, Stickiness, of the dough were evaluated by an experienced baker and judged as good.

Volume, crumb structure and crumb colour of the bread were judged by an experienced baker as good.

Satisfactory results were obtained, that indicated a good dough and a good bread.

After cooling down to room temperature the volumes of the loaves were determined by an automated bread volume analyser (BVM-3, TexVol Instruments). The loaf volume of the control bread is defined as 100%.

The breads were kept in the plastic bags in between the hardness and resilience measurements.

TABLE 1

| | Sponge & Dough |
|---|---|
| Ingredients | (amounts in grams) |
| Sponge | |
| Flour King Arthur USA | 1250 |
| Water | 813 |
| Yeast instant dry | 85 |
| Dough | |
| Flour King Arthur USA | 1250 |
| Water | 828 |
| Yeast instant dry | 18.8 |
| Sugar | 175 |
| Shortening | 100 |
| Salt | 50 |
| Conditioner* | 25 |
| Calcium propionate | 10 |

All ingredients except ascorbic acid and enzymes were supplied by Inter-County Bakers, Inc. Lindenhurst, New York
*Conditioner comprising 50 ppm ascorbic acid (from DSM Nutritional Products, Switzerland), 5 ppm Bakezyme ® P500 (fungal alpha-amylase from DSM, The Netherlands), 20 ppm Bakezyme ® HSP6000 (fungal hemicellulase from DSM, The Netherlands), 20 ppm Bakezyme ® BXP5000 (bacterial hemicellulase from DSM, The Netherlands), 30 wt % EMPLEX (SSL from Caravan Ingredients, USA) 30 wt % STARPLEX 90 (Monoglyceride from Caravan Ingredients, USA), 5 wt % canola oil for anti-dusting (Bunge Oils, USA) and King Arthur flour as mixing material.

Measurement of Hardness and Resilience

The bread was cut in slices of 1 inch or 2.5 cm thickness and the hardness was measured using a Texture Analyser TA-XTPlus from Stable Micro Systems apparatus and applying the following settings.

Settings

Test mode=Compression

Pre-test speed=3 mm/s

Test speed=1 mm/s

Post test speed 5 mm/s

Distance=5 mm

Hold time=10 sec

Trigger force=5 g

The hardness listed is the Force measured; the max peak value recorded in gram. The margin of error may vary within baking trials and usually is smaller than about 10% (smaller than about 40 units of hardness).

Resilience is the Force (F) after 10 sec holding time divided by max peak force multiplied by 100. Resilience=(F2/F1)×100. The margin of error may vary within baking trials and usually is smaller than 10% (smaller than about 0.03 units of resilience).

TABLE 2
Average values with recipe for Sponge and Dough
(average from 2 breads, 9 slices per bread (1
inch = 2.54 cm thick) were measured).

| | Hardness | Hardness | Hardness |
|---|---|---|---|
| | day 1 | day 7 | day 14 |
| Control | 276 | 481 | 597 |
| 50 ppm | 206 | 335 | 448 |
| Mature DSM-AM | | | |
| 85 ppm | 194 | 322 | 421 |
| Mature DSM-AM | | | |
| 57.5 ppm | 224 | 363 | 485 |
| Powerfresh Bread | | | |
| 8100 | | | |
| 115 ppm | 188 | 304 | 426 |
| Powerfresh Bread | | | |
| 8100 | | | |
| 50 ppm | 177 | 281 | 366 |
| mature DSM-AM and | | | |
| 57.5 ppm Powerfresh | | | |
| Bread 8100 | | | |

Day 1 is the first day after the day the bread was baked. Day 7 is the 7th day after the bread was baked. Day 14 is the 14th day after the bread was baked. Activity of Mature DSM-AM used was: 950 NBAU/gram enzyme. ppm means mg/kg, e.g. 50 ppm means 50 mg of the indicated product per kg flour.

TABLE 3
Average values with recipe for Sponge and Dough
(average from 2 breads, 9 slices per bread (1
inch = 2.54 cm thick) were measured).

| | Resilience | Resilience | Resilience |
|---|---|---|---|
| | day 1 | day 7 | day 14 |
| Control | 0.300 | 0.214 | 0.165 |
| 50 ppm | 0.344 | 0.268 | 0.234 |
| Mature DSM-AM | | | |
| 85 ppm | 0.341 | 0.281 | 0.256 |
| Mature DSM-AM | | | |
| 57.5 ppm | 0.322 | 0.256 | 0.227 |
| Powerfresh Bread | | | |
| 8100 | | | |
| 115 ppm | 0.353 | 0.291 | 0.268 |
| Powerfresh Bread | | | |
| 8100 | | | |
| 50 ppm | 0.347 | 0.291 | 0.272 |
| mature DSM-AM and | | | |
| 57.5 ppm Powerfresh | | | |
| Bread 8100 | | | |

Day 1 is the first day after the day the bread was baked. Day 7 is the 7th day after the bread was baked. Day 14 is the 14th day after the bread was baked. Activity of Mature DSM-AM used was: 950 NBAU/gram enzyme. ppm means mg/kg, e.g. 50 ppm means 50 mg of the indicated product per kg flour.

### Example 2

### Baking Experiment, Dose Response Curves

The dose response curve of the mature DSM-AM was tested in American style Sponge and Dough white bread. The ingredients and recipe used are similar as mentioned in Example 1 in this invention, recipe using three amounts of mature DSM-AM: 50 ppm, 75 ppm and 100 ppm, respectively (enzyme having an activity of 750 NBAU/gram enzyme)

The following was observed. On day 1, i.e. the day the bread was baked, the hardness of the three amounts was within each other's error margin. The same was observed on the 8^{th} day after the bread was baked (day 8) and on the 15^{th} day after the bread was baked (day 15).

This illustrates that the reduction in hardness compared to a control reaches a plateau value above the addition of a certain amount of the enzyme product, above this amount no further firmness benefit is observed. This plateau effect on adding more enzyme is not uncommon for such type of enzymes.

For the resilience a similar effect was observed. On day 1, i.e. the day the bread was baked, the resilience of the three amounts was within each other's error margin. The same was observed for day 8 and day 15. This illustrates that the increase in resilience compared to a control reaches a plateau value above adding a certain amount of the enzyme product. It is not uncommon for the application of enzymes in bread that a plateau is reached where a further increase of the enzyme dosage has no additional effect.

### Example 3

### Baking Experiment

Breads were prepared analogous to example 1 using Mature DSM-AM (alpha-amylase polypeptide) and PowerFRESH Special (G4-forming amylase product by DuPont Industrial Biosciences, Denmark).

The baking performance of the mature DSM-AM, PowerFRESH Special (an example of a G-4 forming amylase from Dupont) and a combination of these enzymes was tested in American style Sponge and Dough white bread. The ingredients used are listed in Table 4. The amounts Mature DSM-AM (alpha-amylase polypeptide) and PowerFRESH Special are listed in Table 5. The Control refers to a loaf of bread prepared according to the same recipe while not containing mature DSM-AM, PowerFRESH Special or a combination thereof.

Mature DSM-AM, may be produced as described in not yet published U.S. patent application Ser. No. 13/532,072. Mature DSM-AM may be produced as described in U.S. Pat. No. 8,426,182 B1.

The ingredients of the sponge listed in Table 4 were mixed in a Hobart A-120 mixer with hook agitator for two minutes at speed one, thereafter for three minutes at speed two, to a final sponge temperature of 24° C. Afterwards the sponge was allowed to ferment for 3 hours at 38° C. in a proof box. After this the ingredients of the dough listed in Table 1 were mixed in the same Hobart A-120 mixer with hook agitator for 30 seconds at speed one. After this the sponge was added to the dough and mixed for another two minutes at speed one, followed by another 8 minutes at speed two to optimum gluten development, to a final dough temperature of 27° C. The fully mixed dough was allowed to rest, covered under plastic, for two minutes at room temperature

The dough was divided in pieces of 565 g, rounded and allowed to rest for 5 minutes at room temperature. Afterwards the dough pieces were moulded using a Unic moulder (top 6.5/bottom 6) and the moulded loaves were placed into bread pans and placed in a proofing cabinet at 38° C. at relative humidity of 85% for 85 minutes. The fully proofed dough pieces were placed in a BeCOM oven and baked in 20 minutes at 215° C. Thereafter the breads were taken out of the oven, depanned and placed on a rack to cool for at least 1 hour at ambient temperature, which is typically between 20 and 25° C. After 1-2 hours cooling, the breads were wrapped in polyethylene plastic bags.

The Consistency, Body, Development, Extensibility, Elasticity, Stickiness, of the dough were evaluated by an experienced baker and judged as good.

Volume, crumb structure and crumb colour of the bread were judged by an experienced baker as good.

The breads were stored for 14 days (Day 1 is the first day after the day the bread was baked). On day 14 the breads were sliced and the slices of bread were folded to analyse their foldability.

The foldability was analysed as follows.

A slice of bread was held in two hands and folded by moving one edge of the slice to the opposite edge. This way a folded slice of bread product was obtained having a bended curve in an area located at or close to the center of the slice. The surface of the outside of the bend of folded baked product was visually inspected. The foldability is improved if fewer cracks are observed at or close to the bend. Foldability is improved if the folded baked product has less tendency to break along the bend.

TABLE 4

| | Sponge & Dough |
|---|---|
| Ingredients | (amounts in grams) |
| Sponge | |
| Flour King Arthur USA | 1250 |
| Water | 813 |
| Yeast instant dry | 25 |
| Dough | |
| Flour King Arthur USA | 1250 |
| Water | 828 |
| Yeast instant dry | 18.8 |
| Sugar | 175 |
| Shortening | 100 |
| Salt | 50 |
| Conditioner* | 25 |
| Calcium propionate | 10 |

All ingredients except ascorbic acid and enzymes were supplied by Inter-County Bakers, Inc. Lindenhurst, New York
*Conditioner comprising 50 ppm ascorbic acid (from DSM Nutritional Products, Switzerland), 5 ppm Bakezyme ® P500 (fungal alpha-amylase from DSM, The Netherlands), 20 ppm Bakezyme ® HSP6000 (fungal hemicellulase from DSM, The Netherlands), 20 ppm Bakezyme ® BXP5000 (bacterial hemicellulase from DSM, The Netherlands), 30 wt % EMPLEX (SSL from Caravan Ingredients, USA) 30 wt % STARPLEX 90 (Monoglyceride from Caravan Ingredients, USA), 5 wt % canola oil for anti-dusting (Bunge Oils, USA) and King Arthur flour as mixing material.

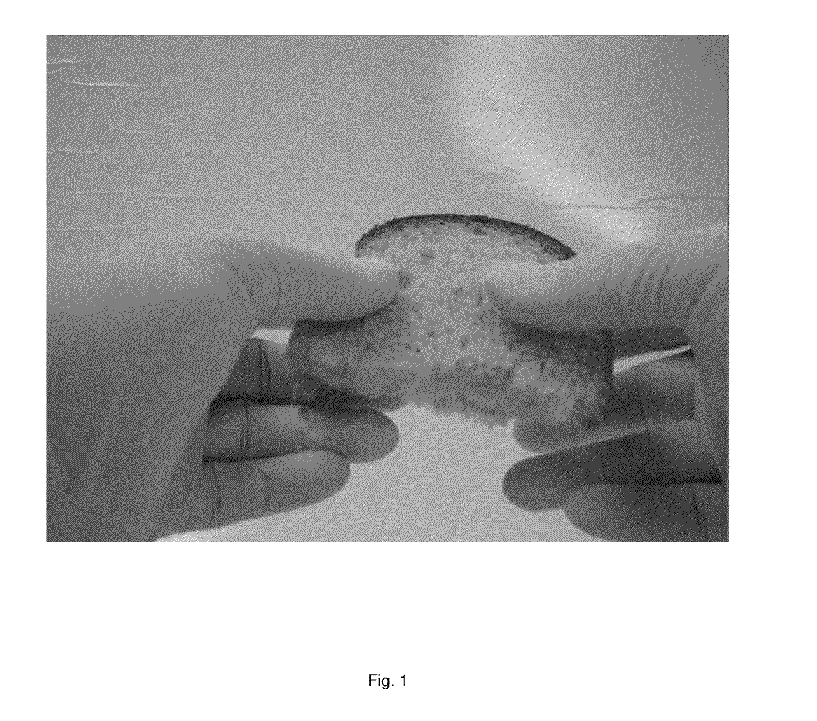

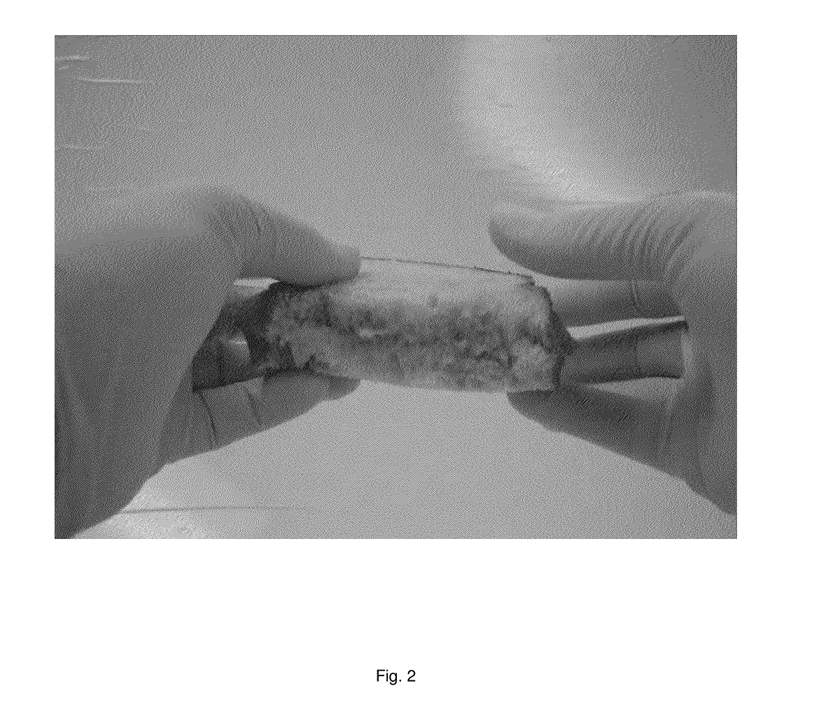

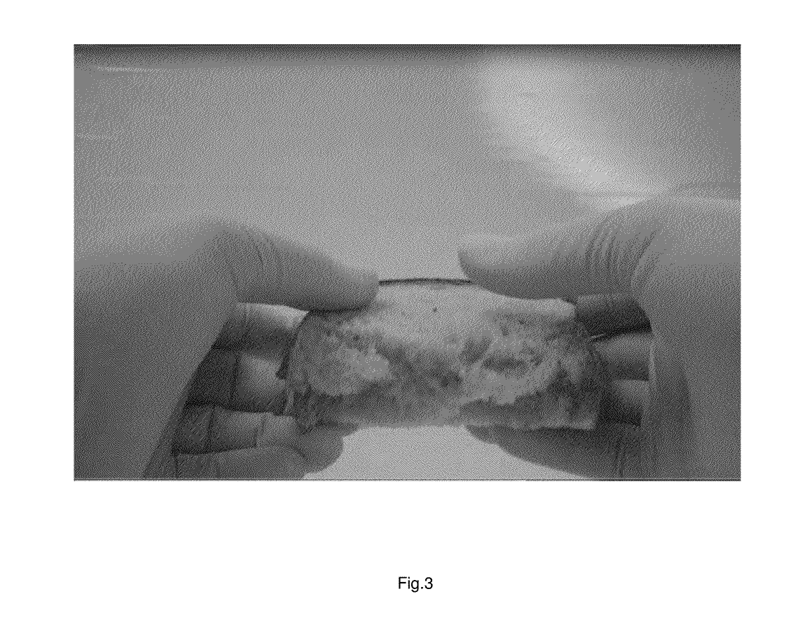

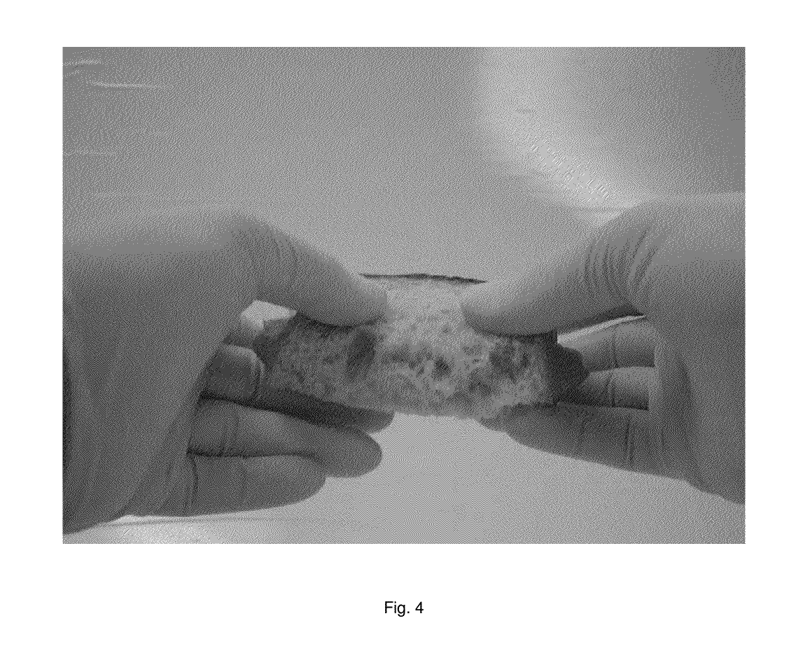

TABLE 5

| | Foldability on Day 14 |
|---|---|
| Control | See FIG. 1 |
| 50 ppm | See FIG. 2 |
| Mature DSM-AM | |
| 75 ppm | See FIG. 3 |
| PowerFRESH Special | |
| 50 ppm | See FIG. 4 |
| mature DSM-AM and | |
| 75 ppm PowerFRESH | |
| Special | |

Day 1 is the first day after the day the bread was baked. Day 7 is the 7th day after the bread was baked. Day 14 is the 14th day after the bread was baked. Activity of Mature DSM-AM used was: 950 NBAU/gram enzyme. ppm means mg/kg, e.g. 50 ppm means 50 mg of the indicated product per kg flour.

The foldability results are shown in FIGS. 1 to 4:

FIG. 1 Photo illustrating foldability of a slice of bread manufactured without Mature DSM-AM and without PowerFRESH Special.

FIG. 2 Photo illustrating of foldability of a slice of bread manufactured using using 50 ppm Mature DSM-AM.

FIG. 3 Photo illustrating of foldability of a slice of bread manufactured using 75 ppm PowerFRESH Special.

FIG. 4 Photo illustrating of foldability of a slice of bread manufactured using using 50 ppm Mature DSM-AM and 75 ppm PowerFRESH Special.

The slices in photo 1 and photo 2 broke on folding. The slice in photo 3 was heavily cracked and nearly broke. The slice in photo 4 was cracked but not broken. FIG. 4 shows the least cracks at surface of the outside of the bend of a folded slice of bread as compared with FIGS. 1 to 3. The slice in photo 4 is clearly the least damaged upon folding.

FIG. 4 therefore illustrates use of an alpha-amylase polypeptide in combination with a G4-forming amylase to improve foldability of a baked product.

1. An enzyme composition comprising an alpha-amylase polypeptide comprising
(a) an amino acid sequence as set out in amino acids 34 to 719 of SEQ ID NO: 2; or
(b) an amino acid sequence having at least 99.5% identity to an amino acid sequence as set out in amino acids 34 to 719 of SEQ ID NO: 2; or
(c) an amino acid sequence encoded by a polynucleotide as set out in nucleotides 100 to 2157 of SEQ ID NO: 1 or SEQ ID NO: 3; or
(d) an amino acid sequence having at least 70% identity to an amino acid sequence as set out in amino acids 34 to 719 of SEQ ID NO: 2 and having at least one of Asp at position 184, Ala at position 297, Thr at position 368 and Asn at position 489, said positions being defined with reference to SEQ ID NO: 2; or
(e) an amino acid sequence having at least 70% identity to an amino acid sequence as set out in amino acids 34 to 719 of SEQ ID NO: 2 and having at least one of Asp at position 184, Ala at position 297, Thr at position 368 and Asn at position 489, said positions being defined with reference to SEQ ID NO: 2 and said amino acid sequence wherein when used to prepare a baked product having a least 5 wt % sugar based on flour, said baked product has reduced hardness after storage in comparison with a baked product prepared without use of said amino acid sequence; or
(f) an amino acid sequence having alpha-amylase activity and having at least 70% identity to an amino acid sequence as set out in amino acids 34 to 719 of SEQ ID NO: 2 and having a substitution, at any one or more positions corresponding to
37, 39, 46, 47, 48, 49, 53, 78, 80, 84, 87, 94, 101, 102, 103, 104, 105, 106, 107, 108, 110, 111, 113, 115, 120, 121, 127, 128, 130, 133, 136, 137, 150, 157, 158, 159, 161, 162, 163, 166, 167, 169, 176, 177, 179, 201, 207, 210, 211, 216, 219, 221, 222, 223, 227, 228, 232, 233, 234, 237, 240, 243, 247, 250, 252, 255, 258, 260, 266, 267, 268, 269, 273, 284, 285, 287, 291, 292, 293, 295, 296, 297, 299, 300, 302, 304, 306, 312, 314, 315, 316, 317, 319, 321, 332, 355, 356, 358, 360, 361, 364, 367, 383, 389, 391, 400, 403, 404, 407, 410, 411, 421, 424, 447, 454, 455, 478, 483, 500, 521, 538, 569, 581, 616, 621, 636, 670, 681, 684, 685, 693, 709, 710,
said positions being defined with reference to an amino acid sequence as set out in amino acids 34 to 719 of SEQ ID NO: 2;
and wherein the composition further comprises a G4-forming amylase having an amino acid sequence at least 70% identical to the amino acid sequence as set out in SEQ ID NO: 4.

2. The enzyme composition according to claim 1, wherein, the alpha-amylase polypeptide comprises an amino acid sequence having alpha-amylase activity and having at least 70% identity with an amino acid sequence as set out in amino acids 34 to 317 of SEQ ID NO: 2, and having a substitution at any one or more positions corresponding to
46, 48, 49, 53, 78, 94, 101, 103, 105, 108, 110, 111, 121, 127, 157, 159, 161, 162, 162, 166, 167, 169, 201, 207, 210, 211, 219, 221, 227, 228, 232, 233, 243, 252, 255, 258, 267, 287, 294, 297, 300, 302, 304, 314, 315, 316, 317, 321, 356, 358, 360, 364, 367, 391, 403, 404, 410, 421, 454, 483, 685,
said positions being defined with reference to an amino acid sequence as set out in amino acids 34 to 719 of SEQ ID NO: 2;
and wherein the alpha-amylase polypeptide preferably demonstrates any one of increased thermostability, or
increased sucrose tolerance, or
increased Activity at pH4: Activity at pH 5 ratio
as compared with a reference polypeptide having an amino acid sequence as set out in amino acids 34 to 317 of SEQ ID NO: 2.

3. The enzyme composition according to claim 1, which comprises an additional enzyme.

4. The enzyme composition according to claim 3 wherein the additional enzyme is selected from the group consisting of an amylase, a further alpha-amylase, beta-amylase, a cyclodextrin glucanotransferase, a protease, a peptidase, a transglutaminase, a triacyl glycerol lipase, a galactolipase, a phospholipase, a cellulase, a hemicellulase, a protease, a protein disulfide isomerase, a glycosyltransferase, a peroxidase, a laccase, an oxidase, a hexose oxidase, a glucose oxidase, a aldose oxidase, a pyranose oxidase, a lipoxygenase, a L-amino acid oxidase and an amyloglucosidase.

5. A pre-mix comprising flour, an alpha-amylase polypeptide comprising (a) an amino acid sequence as set out in amino acids 34 to 719 of SEQ ID NO: 2; or
(b) an amino acid sequence having at least 99.5% identity to an amino acid sequence as set out in amino acids 34 to 719 of SEQ ID NO: 2; or
(c) an amino acid sequence encoded by a polynucleotide as set out in nucleotides 100 to 2157 of SEQ ID NO: 1 or SEQ ID NO: 3; or
(d) an amino acid sequence having at least 70% identity to an amino acid sequence as set out in amino acids 34 to 719 of SEQ ID NO: 2 and having at least one of Asp at position 184, Ala at position 297, Thr at position 368 and Asn at position 489, said positions being defined with reference to SEQ ID NO: 2; or
(e) an amino acid sequence having at least 70% identity to an amino acid sequence as set out in amino acids 34 to 719 of SEQ ID NO: 2 and having at least one of Asp at position 184, Ala at position 297, Thr at position 368 and Asn at position 489, said positions being defined with reference to SEQ ID NO: 2 and said amino acid sequence wherein when used to prepare a baked product having a least 5 wt % sugar based on flour, said baked product has reduced hardness after storage in comparison with a baked product prepared without use of said amino acid sequence; or
(f) an amino acid sequence having alpha-amylase activity and having at least 70% identity to an amino acid sequence as set out in amino acids 34 to 719 of SEQ ID NO: 2 and having a substitution, at any one or more positions corresponding to
37, 39, 46, 47, 48, 49, 53, 78, 80, 84, 87, 94, 101, 102, 103, 104, 105, 106, 107, 108, 110, 111, 113, 115, 120, 121, 127, 128, 130, 133, 136, 137, 150, 157, 158, 159, 161, 162, 163, 166, 167, 169, 176, 177, 179, 201, 207, 210, 211, 216, 219, 221, 222, 223, 227, 228, 232, 233, 234, 237, 240, 243, 247, 250, 252, 255, 258, 260, 266, 267, 268, 269, 273, 284, 285, 287, 291, 292, 293, 295, 296, 297, 299, 300, 302, 304, 306, 312, 314, 315, 316, 317, 319, 321, 332, 355, 356, 358, 360, 361, 364, 367, 383, 389, 391, 400, 403, 404, 407, 410, 411, 421, 424, 447, 454, 455, 478, 483, 500, 521, 538, 569, 581, 616, 621, 636, 670, 681, 684, 685, 693, 709, 710,
said positions being defined with reference to an amino acid sequence as set out in amino acids 34 to 719 of SEQ ID NO: 2;
and a G4-forming amylase having an amino acid sequence at least 70% identical to the amino acid sequence as set out in SEQ ID NO: 4.

6. The pre-mix according to claim 5, wherein the pre-mix further comprises one or more components selected from the group consisting of milk powder, gluten, granulated fat, an additional enzyme, an amino acid, a salt, an oxidant agent optionally comprising ascorbic acid, bromate and azodicarbonamide, a reducing agent optionally comprising L-cysteine, an emulsifier agent optionally comprising mono-glycerides, di-glycerides, sodium stearoyl lactylate, calcium stearoyl lactylate, polyglycerol esters of fatty acids and di acetyl tartaric acid esters of mono- and diglycerides, gums agent optionally comprising guargum and xanthangum, flavours, acids agent optionally comprising citric acid and propionic acid, starch, modified starch, gluten, humectants agent optionally comprising glycerol, and preservatives.

7. The pre-mix according to claim 6, wherein the additional enzyme is selected from the group consisting of an amylase, a further alpha-amylase, beta-amylase, a cyclodextrin glucanotransferase, a protease, a peptidase, a transglutaminase, a triacyl glycerol lipase, a galactolipase, a phospholipase, a cellulase, a hemicellulase, a protease, a protein disulfide isomerase, a glycosyltransferase, a peroxidase, a laccase, an oxidase, a hexose oxidase, a glucose oxidase, a aldose oxidase, a pyranose oxidase, a lipoxygenase, a L-amino acid oxidase and an amyloglucosidase.

8. A method to prepare a dough comprising combining an alpha-amylase polypeptide comprising (a) an amino acid sequence as set out in amino acids 34 to 719 of SEQ ID NO: 2; or
(b) an amino acid sequence having at least 99.5% identity to an amino acid sequence as set out in amino acids 34 to 719 of SEQ ID NO: 2; or
(c) an amino acid sequence encoded by a polynucleotide as set out in nucleotides 100 to 2157 of SEQ ID NO: 1 or SEQ ID NO: 3; or
(d) an amino acid sequence having at least 70% identity to an amino acid sequence as set out in amino acids 34 to 719 of SEQ ID NO: 2 and having at least one of Asp at position 184, Ala at position 297, Thr at position 368 and Asn at position 489, said positions being defined with reference to SEQ ID NO: 2; or
(e) an amino acid sequence having at least 70% identity to an amino acid sequence as set out in amino acids 34 to 719 of SEQ ID NO: 2 and having at least one of Asp at position 184, Ala at position 297, Thr at position 368 and Asn at position 489, said positions being defined with reference to SEQ ID NO: 2 and said amino acid sequence wherein when used to prepare a baked product having a least 5 wt % sugar based on flour, said baked product has reduced hardness after storage in comparison with a baked product prepared without use of said amino acid sequence; or
(f) an amino acid sequence having alpha-amylase activity and having at least 70% identity to an amino acid sequence as set out in amino acids 34 to 719 of SEQ ID NO: 2 and having a substitution, at any one or more positions corresponding to
37, 39, 46, 47, 48, 49, 53, 78, 80, 84, 87, 94, 101, 102, 103, 104, 105, 106, 107, 108, 110, 111, 113, 115, 120, 121, 127, 128, 130, 133, 136, 137, 150, 157, 158, 159, 161, 162, 163, 166, 167, 169, 176, 177, 179, 201, 207, 210, 211, 216, 219, 221, 222, 223, 227, 228, 232, 233, 234, 237, 240, 243, 247, 250, 252, 255, 258, 260, 266, 267, 268, 269, 273, 284, 285, 287, 291, 292, 293, 295, 296, 297, 299, 300, 302, 304, 306, 312, 314, 315, 316, 317, 319, 321, 332, 355, 356, 358, 360, 361, 364, 367, 383, 389, 391, 400, 403, 404, 407, 410, 411, 421, 424, 447, 454, 455, 478, 483, 500, 521, 538, 569, 581, 616, 621, 636, 670, 681, 684, 685, 693, 709, 710,
said positions being defined with reference to an amino acid sequence as set out in amino acids 34 to 719 of SEQ ID NO: 2;
and
a G4-forming amylase having an amino acid sequence at least 70% identical to the amino acid sequence as set out in SEQ ID NO: 4, and
at least one dough ingredient.

9. A method to prepare a dough comprising combining an enzyme composition according to claim 1, with at least one dough ingredient.

10. A dough comprising the pre-mix according to claim 5.

11. Method to prepare a baked product comprising baking the dough according to claim 10.

12. Baked product obtainable by the method according to claim 11.

13. An alpha-amylase polypeptide comprising (a) an amino acid sequence as set out in amino acids 34 to 719 of SEQ ID NO: 2; or
(b) an amino acid sequence having at least 99.5% identity to an amino acid sequence as set out in amino acids 34 to 719 of SEQ ID NO: 2; or
(c) an amino acid sequence encoded by a polynucleotide as set out in nucleotides 100 to 2157 of SEQ ID NO: 1 or SEQ ID NO: 3; or
(d) an amino acid sequence having at least 70% identity to an amino acid sequence as set out in amino acids 34 to 719 of SEQ ID NO: 2 and having at least one of Asp at position 184, Ala at position 297, Thr at position 368 and Asn at position 489, said positions being defined with reference to SEQ ID NO: 2; or
(e) an amino acid sequence having at least 70% identity to an amino acid sequence as set out in amino acids 34 to 719 of SEQ ID NO: 2 and having at least one of Asp at position 184, Ala at position 297, Thr at position 368 and Asn at position 489, said positions being defined with reference to SEQ ID NO: 2 and said amino acid sequence wherein when used to prepare a baked product having a least 5 wt % sugar based on flour, said baked product has reduced hardness after storage in comparison with a baked product prepared without use of said amino acid sequence; or
(f) an amino acid sequence having alpha-amylase activity and having at least 70% identity to an amino acid sequence as set out in amino acids 34 to 719 of SEQ ID NO: 2 and having a substitution, at any one or more positions corresponding to 37, 39, 46, 47, 48, 49, 53, 78, 80, 84, 87, 94, 101, 102, 103, 104, 105, 106, 107, 108, 110, 111, 113, 115, 120, 121, 127, 128, 130, 133, 136, 137, 150, 157, 158, 159, 161, 162, 163, 166, 167, 169, 176, 177, 179, 201, 207, 210, 211, 216, 219, 221, 222, 223, 227, 228, 232, 233, 234, 237, 240, 243, 247, 250, 252, 255, 258, 260, 266, 267, 268, 269, 273, 284, 285, 287, 291, 292, 293, 295, 296, 297, 299, 300, 302, 304, 306, 312, 314, 315, 316, 317, 319, 321, 332, 355, 356, 358, 360, 361, 364, 367, 383, 389, 391, 400, 403, 404, 407, 410, 411, 421, 424, 447, 454, 455, 478, 483, 500, 521, 538, 569, 581, 616, 621, 636, 670, 681, 684, 685, 693, 709, 710,
said positions being defined with reference to an amino acid sequence as set out in amino acids 34 to 719 of SEQ ID NO: 2 and capable of being used in combination with a G4-forming amylase having an amino acid sequence at least 70% identical to the amino acid sequence as set out in SEQ ID NO: 4, to reduce hardness after storage of a baked and/or to reduce loss of resilience over storage of a baked product.

14. An enzyme composition according to claim 1 capable of being used to reduce hardness after storage of a baked product and/or to reduce loss of resilience over storage of a baked product.

15. An alpha-amylase polypeptide comprising (a) an amino acid sequence as set out in amino acids 34 to 719 of SEQ ID NO: 2; or
(b) an amino acid sequence having at least 99.5% identity to an amino acid sequence as set out in amino acids 34 to 719 of SEQ ID NO: 2; or
(c) an amino acid sequence encoded by a polynucleotide as set out in nucleotides 100 to 2157 of SEQ ID NO: 1 or SEQ ID NO: 3; or
(d) an amino acid sequence having at least 70% identity to an amino acid sequence as set out in amino acids 34 to 719 of SEQ ID NO: 2 and having at least one of Asp at position 184, Ala at position 297, Thr at position 368 and Asn at position 489, said positions being defined with reference to SEQ ID NO: 2; or
(e) an amino acid sequence having at least 70% identity to an amino acid sequence as set out in amino acids 34 to 719 of SEQ ID NO: 2 and having at least one of Asp at position 184, Ala at position 297, Thr at position 368 and Asn at position 489, said positions being defined with reference to SEQ ID NO: 2 and said amino acid sequence wherein when used to prepare a baked product having a least 5 wt % sugar based on flour, said baked product has reduced hardness after storage in comparison with a baked product prepared without use of said amino acid sequence; or
(f) an amino acid sequence having alpha-amylase activity and having at least 70% identity to an amino acid sequence as set out in amino acids 34 to 719 of SEQ ID NO: 2 and having a substitution, at any one or more positions corresponding to 37, 39, 46, 47, 48, 49, 53, 78, 80, 84, 87, 94, 101, 102, 103, 104, 105, 106, 107, 108, 110, 111, 113, 115, 120, 121, 127, 128, 130, 133, 136, 137, 150, 157, 158, 159, 161, 162, 163, 166, 167, 169, 176, 177, 179, 201, 207, 210, 211, 216, 219, 221, 222, 223, 227, 228, 232, 233, 234, 237, 240, 243, 247, 250, 252, 255, 258, 260, 266, 267, 268, 269, 273, 284, 285, 287, 291, 292, 293, 295, 296, 297, 299, 300, 302, 304, 306, 312, 314, 315, 316, 317, 319, 321, 332, 355, 356, 358, 360, 361, 364, 367, 383, 389, 391, 400, 403, 404, 407, 410, 411, 421, 424, 447, 454, 455, 478, 483, 500, 521, 538, 569, 581, 616, 621, 636, 670, 681, 684, 685, 693, 709, 710,
said positions being defined with reference to an amino acid sequence as set out in amino acids 34 to 719 of SEQ ID NO: 2 in combination with a G4-forming amylase having an amino acid sequence at least 70% identical to the amino acid sequence as set out in SEQ ID NO: 4, capable of being used to improve foldability of a baked product.

16. A pre-mix according to claim 5 capable of being used to improve foldability of a baked product.


SEQUENCE LISTING (5 sequences)

SEQ ID NO: 1 - DNA, 2160 bp, Alicyclobacillus pohliae
atgaaaaagaaaacgctttcattatttgtgggactgatgctgctcctcggtcttctgttcagcggttctcttccgtacaatccaaacgccgctgaagccagcagttccgcaagcgtcaaaggggacgtgatttaccagattatcattgaccggttttacgatggggacacgacgaacaacaatcctgccaaaagttatggactttacgatcccaccaaatcgaagtggaaaatgtattggggcggggatctggagggggttcgtcaaaaacttccttatcttaaacagctgggcgtaacgacgatctggttgtccccggttttggacaatctggatacacttgcaggtaccgataacactggctatcacggatactggacgcgcgattttaaacagattgaggaacatttcgggaattggaccacatttgacacgttggtcaatgatgctcaccaaaacggaatcaaggtgattgtcgactttgtgcccaatcattcaactccttttaaggcaaacgattccacctttgcggaaggcggcgccctctacgacaacggaacctatatgggcaattattttgatgacgcaacaaaagggtactttcaccataatggggacatcagcaactgggacgaccggtacgaggcgcaatggaaaaacttcacggatccagccggtttctcgcttgccgatttgtcgcaggaaaatggcacgattgctcaatacctgaccgatgcggcggttcaattagtagcacatggagcggatggtttgcggattgatgcggtgaagcattttaattctgggttctccaaatcgttggctgataaactgtaccaaaagaaagacattttcctagtgggggaatggtacggagatgaccccggagcagccaatcatttggaaaaggtccggtacgccaacaacagcggtgtcaatgtgctggattttgatctcaacacggtgattcgaaatgtgttcggtacatttacgcaaacgatgtacgatcttaacaatatggtgaaccaaacggggaacgagtacaaatacaaagaaaatctaatcacatttatcgataaccatgatatgtcgagatttcttacggtaaattcgaacaaggcgaatttgcaccaggcgcttgctttcattctcacttcgcggggaacgccctccatctattacggaaccgaacaatacatggcaggcggcaatgacccgtacaacagggggatgatgccggcgtttgatacgacaaccaccgcctttaaagaggtgtcaactctggcggggttgcgcaggaacaatgcagcgatccagtacggcaccaccacccaacgttggatcaacaatgatgtttacatttatgagcggaaatttttcaacgatgtcgtattggtggccatcaatcgaaacacgcaatcctcctactcgatttccggtttgcagactgccttgccaaatggcaactatgcggattatctgtcagggctgttgggggggaacgggatttccgtttccaatggaagtgtcgcttcgttcacgcttgcgcctggagccgtgtctgtttggcagtacagcacatccgcttcagcgccgcaaatcggatcggttgctccgaatatgggaattccgggtaatgtggtcacgatcgacgggaaaggttttggaacgacgcagggaaccgtgacatttggcggagtgacagcgactgtaaaatcctggacatcaaaccggattgaagtgtacgtgcccaacatggccgccggtctgaccgatgtaaaagtcaccgcgggtggagtttccagcaatctgtattcttacaatattttgagtggaacgcagacatcggttgtgtttactgtgaaaagtgctcctccgaccaacctgggggataagatttacctgacgggcaacataccggaattgggaaattggagcacggatacgagcggagccgttaacaatgcgcaagggcccctgctcgcgcccaattatccggattggttttatgtattcagcgttccggcaggaaagacgattcaattcaagtttttcatcaagcgtgcggatggaacgattcaatgggagaatggttcgaaccacgtggccacaactcccacgggtgcaaccggtaacatcactgtcacgtggcaaaactag

SEQ ID NO: 2 - protein, 719 aa, Alicyclobacillus pohliae
MKKKTLSLFVGLMLLLGLLFSGSLPYNPNAAEASSSASVKGDVIYQIIIDRFYDGDTTNNNPAKSYGLYDPTKSKWKMYWGGDLEGVRQKLPYLKQLGVTTIWLSPVLDNLDTLAGTDNTGYHGYWTRDFKQIEEHFGNWTTFDTLVNDAHQNGIKVIVDFVPNHSTPFKANDSTFAEGGALYDNGTYMGNYFDDATKGYFHHNGDISNWDDRYEAQWKNFTDPAGFSLADLSQENGTIAQYLTDAAVQLVAHGADGLRIDAVKHFNSGFSKSLADKLYQKKDIFLVGEWYGDDPGAANHLEKVRYANNSGVNVLDFDLNTVIRNVFGTFTQTMYDLNNMVNQTGNEYKYKENLITFIDNHDMSRFLTVNSNKANLHQALAFILTSRGTPSIYYGTEQYMAGGNDPYNRGMMPAFDTTTTAFKEVSTLAGLRRNNAAIQYGTTTQRWINNDVYIYERKFFNDVVLVAINRNTQSSYSISGLQTALPNGNYADYLSGLLGGNGISVSNGSVASFTLAPGAVSVWQYSTSASAPQIGSVAPNMGIPGNVVTIDGKGFGTTQGTVTFGGVTATVKSWTSNRIEVYVPNMAAGLTDVKVTAGGVSSNLYSYNILSGTQTSVVFTVKSAPPTNLGDKIYLTGNIPELGNWSTDTSGAVNNAQGPLLAPNYPDWFYVFSVPAGKTIQFKFFIKRADGTIQWENGSNHVATTPTGATGNITVTWQN

SEQ ID NO: 3 - DNA, 2160 bp, Artificial Sequence
  Sets out the artificial codon pair optimized polynucleotide sequence from Alicyclobacillus pohliae NCIMB14276 encoding the alpha-amylase according to the invention
atgaagaagaaaacactttctctatttgtcggtttgatgctgctgcttggtttgctgttctctggttcacttccttacaacccgaatgcagctgaggcttcttcaagtgcaagtgtgaagggagatgtgatttaccaaatcatcatcgaccgtttctatgacggtgacacaacaaacaacaatccggcaaaatcatacggcctgtatgatccgacaaaaagcaaatggaaaatgtactggggcggagatcttgaaggcgttcgccaaaagctgccatatttgaagcagcttggtgtaacgacgatttggctttcgcctgttcttgacaatcttgatacgctggcaggtactgacaatacaggttatcacggctactggacaagagatttcaaacaaatcgaagagcatttcggaaactggacgacatttgacacacttgtgaatgatgctcaccaaaacggcatcaaagtgatcgttgatttcgttccgaatcacagcacgccattcaaagcaaacgacagcacgtttgcagaaggcggtgctttgtacgataacggtacttacatgggaaattattttgatgatgcaacaaaaggctatttccatcataacggagatatcagcaactgggatgaccgttatgaagcacaatggaaaaacttcacagatcctgctggcttcagccttgctgatttatcacaagaaaacggaacgatcgctcaatatttaactgacgctgctgttcagcttgttgctcacggtgctgacggccttcgcattgatgcagtgaagcacttcaacagcggcttcagcaaaagccttgctgacaagctgtatcaaaagaaggatattttccttgtcggtgaatggtatggagatgacccaggtgctgctaatcaccttgaaaaagtgcgttatgcaaacaactctggtgtaaatgtgcttgattttgatttgaatacggttatccgcaatgtattcggaacatttacacaaacgatgtacgatttaaacaacatggtgaaccaaacaggaaatgaatacaaatataaagaaaacctgattacatttattgacaaccatgatatgagccgcttcctgactgtaaacagcaacaaagcaaaccttcatcaggcacttgcttttattttaacttcaagaggaacaccgtcaatttactacggaacagaacaatatatggcaggcggaaatgatccatacaaccgcggcatgatgcctgcttttgatacaacaacaactgcattcaaagaagtatcaacgcttgcagggctgcgtcgtaataatgcagcaattcaatacggcacaacaactcagcgctggatcaacaatgatgtatacatatatgaaagaaaattctttaatgatgttgtgcttgttgcaatcaaccgaaatacacaatcttcttattccatcagcggccttcaaacggcactgccaaacggaaactacgctgattacctttccggcctgcttggcggaaacggaatttctgtcagcaacggttctgttgcatcatttacgcttgctcctggtgctgtttctgtttggcaatattcaacttcagcttctgctcctcaaatcggttctgttgcaccgaatatgggtatcccgggaaacgttgtgacgattgacggaaaaggcttcggaacgacacaaggtactgtaacattcggcggcgttactgcaactgtaaaaagctggacatcaaaccgtattgaagtgtatgtgccgaatatggctgctggcctgactgatgtaaaagtgacagctggcggtgtttcttcaaacctatactcttacaacattttatcaggcacacaaacatctgttgtattcactgtaaaatcagcaccgccgacaaacctaggtgacaagatttacttaacaggaaacatccctgagcttggaaactggagcactgatacaagcggagctgttaacaatgcacaaggcccgcttcttgcaccgaattatccggactggttttatgtattctctgttcctgctggaaaaacgattcaattcaaattctttatcaaacgcgctgacggaacgattcaatgggaaaacggttcaaaccatgtggcaacaactccaactggtgcaacaggaaatatcactgttacttggcagaattaa

SEQ ID NO: 4 - protein, 429 aa, Pseudomonas saccharophilia
DQAGKSPAGVRYHGGDEIILQGFHWNVVREAPNDWYNILRQQASTIAADGFSAIWMPVPWRDFSSWTDGGKSGGGEGYFWHDFNKNGRYGSDAQLRQAAGALGGAGVKVLYDVVPNHMNRGYPDKEINLPAGQGFWRNDCADPGNYPNDCDDGDRFIGGESDLNTGHPQIYGMFRDELANLRSGYGAGGFRFDFVRGYAPERVDSWMSDSADSSFCVGELWKGPSEYPSWDWRNTASWQQIIKDWSDRAKCPVFDFALKERMQNGSVADWKHGLNGNPDPRWREVAVTFVDNHDTGYSPGQNGGQHHWALQDGLIRQAYAYILTSPGTPVVYWSHMYDWGYGDFIRQLIQVRRTAGVRADSAISFHSGYSGLVATVSGSQQTLVVALNSDLANPGQVASGSFSEAVNASNGQVRVWRSGSGDGGGNDGG

SEQ ID NO: 5 - protein, 429 aa, Artificial Sequence
  Synthetic polypeptide
DQAGKSPAGVRYHGGDEIILQGFHWNVVREAPYNWYNILRQQASTIAADGFSAIWMPVPWRDFSSWTDGDKSGGGEGYFWHDFNKNGRYGSDAQLRQAAGALGGAGVKVLYDVVPNHMNRFYPDKEINLPAGQRFWRNDCPDPGNGPNDCDDGDRFLGGEADLNTGHPQIYGMFRDEFTNLRSGYGAGGFRFDFVRGYAPERVDSWMSDSADSSFCVGELWKEPSEYPPWDWRNTASWQQIIKDWSDRAKCPVFDFALKERMQNGSVADWKHGLNGNPDPRWREVAVTFVDNHDTGYSPGQNGGQHKWPLQDGLIRQAYAYILTSPGTPVVYWPHMYDWGYGDFIRQLIQVRRTAGVRADSAISFHSGYSGLVATVSGSQQTLVVALNSDLANPGQVASGSFSEAVNASNGQVRVWRSGSGDGGGNDGG